SEQUENCE LISTING (44 sequences)

SEQ ID NO: 1 - protein, 7 aa, synthetic construct
GFAFSNY

SEQ ID NO: 2 - protein, 6 aa, synthetic construct
FPGDGD

SEQ ID NO: 3 - skipped

SEQ ID NO: 4 - protein, 25 aa, synthetic construct
QVQLQQSGAELVRPGSSVKISCKAS

SEQ ID NO: 5 - protein, 19 aa, synthetic construct
WMNWVKQRPGQGLEWIGQI

SEQ ID NO: 6 - protein, 41 aa, synthetic construct
TNYNGKFKGKATLTADKSSSTAYMQLISLTSEDSAVYFCVR

SEQ ID NO: 7 - protein, 11 aa, synthetic construct
WGRGTTLTVSS

SEQ ID NO: 8 - protein, 112 aa, synthetic construct
QVQLQQSGAELVRPGSSVKISCKASGFAFSNYWMNWVKQRPGQGLEWIGQIFPGDGDTNYNGKFKGKATLTADKSSSTAYMQLISLTSEDSAVYFCVRESDWGRGTTLTVSS

SEQ ID NO: 9 - protein, 11 aa, synthetic construct
SASQGINNYLN

SEQ ID NO: 10 - protein, 7 aa, synthetic construct
YTSSLQS

SEQ ID NO: 11 - protein, 9 aa, synthetic construct
QQYSKLPFT

SEQ ID NO: 12 - protein, 23 aa, synthetic construct
DIQMTQTTSSLSASLGDRVTISC

SEQ ID NO: 13 - protein, 15 aa, synthetic construct
WYQQTPDGTVKLLIF

SEQ ID NO: 14 - protein, 32 aa, synthetic construct
GVPSRFSGSGSGTDYSLTISNLEPEDIATYYC

SEQ ID NO: 15 - protein, 11 aa, synthetic construct
FGSGTKLEIKR

SEQ ID NO: 16 - protein, 108 aa, synthetic construct
DIQMTQTTSSLSASLGDRVTISCSASQGINNYLNWYQQTPDGTVKLLIFYTSSLQSGVPSRFSGSGSGTDYSLTISNLEPEDIATYYCQQYSKLPFTFGSGTKLEIKR

SEQ ID NO: 17 - protein, 13 aa, synthetic construct
KASGYSFTVYYMN

SEQ ID NO: 18 - protein, 10 aa, synthetic construct
RIDPNDGGAS

SEQ ID NO: 19 - protein, 11 aa, synthetic construct
ARRDYYGSKDY

SEQ ID NO: 20 - protein, 22 aa, synthetic construct
EVQLQQSGPDLVKPGASVKISC

SEQ ID NO: 21 - protein, 14 aa, synthetic construct
WVKQSHGKSLEWIG

SEQ ID NO: 22 - protein, 37 aa, synthetic construct
YNQKFKGKAILTVDKSSGTAYMELRSLTSEDSAVYYC

SEQ ID NO: 23 - protein, 11 aa, synthetic construct
WGQGTTLTVSS

SEQ ID NO: 24 - protein, 118 aa, synthetic construct
EVQLQQSGPDLVKPGASVKISCKASGYSFTVYYMNWVKQSHGKSLEWIGRIDPNDGGASYNQKFKGKAILTVDKSSGTAYMELRSLTSEDSAVYYCARRDYYGSKDYWGQGTTLTVSS

SEQ ID NO: 25 - protein, 15 aa, synthetic construct
KASQSVDYDGDSYMN

SEQ ID NO: 26 - protein, 8 aa, synthetic construct
YAASNLES

SEQ ID NO: 27 - protein, 9 aa, synthetic construct
QQSNEDPWT

SEQ ID NO: 28 - protein, 23 aa, synthetic construct
DIVLTQSPASLAVSLGQRATISC

SEQ ID NO: 29 - protein, 14 aa, synthetic construct
WYQQKPGQPPKLLI

SEQ ID NO: 30 - protein, 32 aa, synthetic construct
GIPARFSGSGSGTDFTLNIHPVEEEDAAIYYC

SEQ ID NO: 31 - protein, 11 aa, synthetic construct
FGGGTKLEIKR

SEQ ID NO: 32 - protein, 112 aa, synthetic construct
DIVLTQSPASLAVSLGQRATISCKASQSVDYDGDSYMNWYQQKPGQPPKLLIYAASNLESGIPARFSGSGSGTDFTLNIHPVEEEDAAIYYCQQSNEDPWTFGGGTKLEIKR

SEQ ID NO: 33 - protein, 684 aa, Homo sapiens
QLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEAN

SEQ ID NO: 34 - protein, 47 aa, Homo sapiens
METDTLLLWVLLLWVPGSTGAGSIBTCPPCPAPELLGGPSVFLFPPJ

SEQ ID NO: 35 - protein, 5 aa, synthetic construct
  VARIANT 1..5: note = The entire sequence of amino acids 1-5 can be repeated three to five times
SGGGG

SEQ ID NO: 36 - protein, 5 aa, synthetic construct
  VARIANT 1..5: note = The entire sequence of amino acids 1-5 can be repeated two to five times.
SGGGG

SEQ ID NO: 37 - DNA, 20 bp, synthetic construct
  source 1..20: mol_type = other DNA
cgagcgcggctctgctcctg

SEQ ID NO: 38 - DNA, 20 bp, synthetic construct
  source 1..20: mol_type = other DNA
agagacctggagacaccagg

SEQ ID NO: 39 - DNA, 20 bp, synthetic construct
  source 1..20: mol_type = other DNA
gggccctttaagaaggttga

SEQ ID NO: 40 - DNA, 20 bp, synthetic construct
  source 1..20: mol_type = other DNA
gttttgacagcccaaaccag

SEQ ID NO: 41 - DNA, 336 bp, synthetic construct
  source 1..336: mol_type = other DNA
caggttcagctgcagcagtctggggctgaactggtgaggcctgggtcctcagtgaagatttcctgcaaggcttctggctttgcattcagtaactactggatgaactgggtgaagcagaggcctggacagggtcttgagtggattggacagatttttcctggagatggtgatactaactacaatggaaagttcaagggtaaagccacactgactgcagacaaatcctccagcacagcctacatgcagctcattagcctaacatctgaggactctgcggtctatttctgtgttagagagagcgactggggccgaggcaccacgctcacagtctcctca

SEQ ID NO: 42 - DNA, 324 bp, synthetic construct
  source 1..324: mol_type = other DNA
gatatccagatgacacagactacatcctccctgtctgcctctctgggagacagagtcaccatcagttgcagtgcaagtcagggcattaacaattatttaaactggtatcagcagacaccagatggaactgttaaactcctgatcttttacacatcaagtttacagtcaggagtcccatcaaggttcagtggcagtgggtctgggacagattattctctcaccatcagcaacctggaacctgaagatattgccacttactattgtcagcagtatagtaagcttccattcacgttcggctcggggacaaagttggaaataaaacgg

SEQ ID NO: 43 - DNA, 354 bp, synthetic construct
  source 1..354: mol_type = other DNA
gaggtccagctgcagcagtctggacctgacctggtgaagcctggggcttcagtgaagatatcctgcaaggcttctggttactcattcactgtctactacatgaactgggtgaagcagagccatggaaagagccttgagtggattggacgtattgatcctaacgatggtggtgctagttacaaccagaagttcaagggcaaggccatattaactgttgacaagtcatccggcacagcctacatggagctccgcagcctgacatctgaggactctgcggtctattactgtgcaagaagggattactacggtagtaaggactactggggccaaggcaccactctcacagtctcctca

SEQ ID NO: 44 - DNA, 336 bp, synthetic construct
  source 1..336: mol_type = other DNA
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctggtatcccagccaggtttagtggcagtgggtctgggacagacttcaccctcaacatccatcctgtggaggaggaggatgctgcaatctattactgtcagcaaagtaatgaggatccgtggacgttcggtggaggcaccaagctggaaatcaaacgg

(19) United States
(12) Patent Application Publication
(10) Pub. No.: US 2025/0313648 A1
(43) Pub. Date: Oct. 9, 2025

(54) Binders of Chondroitin Sulfate Proteoglycan (CSPG4) Polypeptides

(71) Applicant: Regents of the University of Minnesota, Minneapolis, MN (US)

(72) Inventors: James Benjamin McCarthy, Minneapolis, MN (US); Jianbo Yang, Minneapolis, MN (US); Matthew Price, Minneapolis, MN (US); Martin Felices, Minneapolis, MN (US); Jeffrey Miller, Minneapolis, MN (US); Todd Lenvik, Minneapolis, MN (US)

(21) Appl. No.: 18/717,696

(22) PCT Filed: Dec. 7, 2022
(86) PCT No.: PCT/US2022/052099

§ 371 Date: Jun. 7, 2024

Related U.S. Application Data
(60) Provisional application No. 63/286,719, filed on Dec. 7, 2021.

(51) Int. Cl. C07K 16/30; C07K 14/725; C07K 16/28

(52) U.S. Cl. CPC C07K 16/3053; C07K 14/7051; C07K 16/2809; C07K 16/283; C07K 2317/31; C07K 2317/565; C07K 2317/622; C07K 2317/732; C07K 2317/76

(57) ABSTRACT

This document provides methods and materials involved in binding a binder (e.g., an antibody, antigen binding fragment, antibody domain, CAR, cell engager, and/or ADC) to a CSPG4 polypeptide. For example, binders (e.g., antibodies, antigen binding fragments, antibody domains, CARs, cell engagers, and/or ADCs) that bind to a CSPG4 polypeptide and methods and materials for using one or more such binding molecules to treat a mammal (e.g., a human) having cancer are provided.

### CROSS-REFERENCE TO RELATED APPLICATIONS

This application claims the benefit of U.S. Patent Application Ser. No. 63/286,719, filed on Dec. 7, 2021. The disclosure of the prior application is considered part of (and is incorporated by reference in) the disclosure of this application.

### STATEMENT OF FEDERALLY SPONSORED RESEARCH

This invention was made with government support under CA111412 and CA197292, awarded by the National Institutes of Health. The government has certain rights in the invention.

### SEQUENCE LISTING

This application contains a Sequence Listing that has been submitted electronically as an XML file named “09531-0491WO1_SL.XML.” The XML file, created on Nov. 17, 2022, is 41000 bytes in size. The material in the XML file is hereby incorporated by reference in its entirety.

### BACKGROUND

### 1. Technical Field

This document relates to methods and materials involved in binding a molecule (e.g., an antibody, a fragment of an antibody, an antibody domain, a chimeric antigen receptor (CAR), a cell engager, or an antibody-drug conjugate (ADC)) to a chondroitin sulfate proteoglycan 4 (CSPG4) polypeptide. For example, this document provides binders (e.g., antibodies, antigen binding fragments, antibody domains, CARs, cell engagers, or ADCs) that bind to a CSPG4 polypeptide and methods and materials for using such binders to treat cancer. This document also provides cells (e.g., host cells) designed to express one or more binders (e.g., antibodies, antigen binding fragments, antibody domains, CARs, or cell engagers) having the ability to bind to a CSPG4 polypeptide and methods and materials for using such cells to treat cancer.

### 2. Background Information

Epithelial ovarian cancer (EOC) is a highly heterogeneous disease that includes a wide spectrum of distinct molecular subtypes and clinical entities. There is a complex basis for interpatient and intrapatient genetic heterogeneity in EOC that is reflected by the distinct genetic signatures associated with different histologic subtypes or genetic/epigenetic changes induced by external stressors such as chemotherapies. See, e.g., Moffitt, et al., Int. J Mol Sci., 20(6):1466 (2019). Although most EOC patients initially respond well to surgical debulking and adjuvant chemotherapy, the occurrence of chemo-resistance is a major hurdle, with 75% of patients experiencing a relapse within five years. See, e.g., Kroeger, et al., Curr Opin Obstet Gynecol., 29(1):26-34 (2017); Lengyel, Am J Pathol., 177(3):1053-64 (2010). Malignant progression also involves extensive intra-tumoral phenotypic heterogeneity related to dynamic biological requirements at different stages in progression. See, Lengyel, 2010, supra; Testa, et al., Medicines (Basel), 5(1):16 (2018); and Moffitt, 2019, supra. These dynamics include localized changes in growth factors and an actively remodeling tumor-associated extracellular matrix which contributes directly to epigenetic changes associated with malignant progression. Additional factors include the presence of therapy-resistant cancer stem cells, the survival of cells within spatially distinct fibrotic or hypoxic microenvironments, and the expansion of mutational variants with increased invasive and/or metastatic potential. See, e.g., Habyan, et al., Oncogene, 37(37):5127-35 (2018); Meads, et al., Nat Rev Cancer, 9(9):665-74 (2009); and Paullin, et al., PLoS One, 12(8):e0182930 (2017). The complex and dynamic mechanisms that impact this extensive intra-tumoral phenotypic heterogeneity have hindered the identification of effective prognostic and predictive biomarkers that can be effectively targeted in patients with EOC.

Ovarian carcinoma metastasis largely occurs via an intraperitoneal (IP) route and is thus distinct from other common carcinomas such as breast and prostate, which primarily utilize the vasculature or lymphatics. See, e.g., Kroeger et al., 2017 supra; Lengyel, 2010 supra; and Habyan et al., 2018 supra. In EOC, individual cells or cell aggregates dissociate from primary tumors to form multicellular spheroids responsible for peritoneal spread, metastasis, and recurrence. See, e.g., Shield, et al., Gynecol Oncol., 113(1):143-8 (2009). The survival of individual cells that give rise to spheroids is facilitated by their anchorage independence and initial resistance to anoikis. Multiple cell adhesion related pathways (e.g., integrins, cadherins, and claudins) contribute to strengthening and compaction of these multicellular aggregates and to their attachment to the mesothelial cells lining multiple organ sites (e.g., omentum) within the pelvis and peritoneum. Increased compaction of cells within spheroids can lead to increased therapy resistance, in part by limiting penetration of chemotherapies into more centrally located cells within these spheroids. See, e.g., Habyan et al., 2018 supra; and Shield, et al., 2009 supra. Their subsequent invasion into the sub-mesothelial tissues involves stimulation by growth factors and chemokines within the microenvironment and activation of tumor associated matrix metalloproteinases, which degrade the underlying extracellular matrices.

As with other cancers, malignant progression in EOC tumor cells is associated with a phenotypic shift from an epithelial to a mesenchymal phenotype (EMT). EMT programs are impacted by multiple and complex mechanisms, which include multiple signaling pathways (e.g., multiple growth factors, Wnt/β-catenin, and Notch) and changes in expression/function of multiple adhesion receptors (E-cadherin/N-cadherin, claudins, and integrins). See, for example, Deng, et al., Oncotarget, 7(34):55771-88 (2016); and Yang, et al., Nat Rev Mol Cell Biol., 21(6):341-52 (2020). Tumor cell detachment from the primary tumor and subsequent spheroid formation has been linked to increased expression of specific mesenchymal transcription factors such as ZEB1 and Slug (Snail2). Mesenchymal transition in cancer is often associated with cancer cell ‘stemness’, resistance to apoptosis, and therapy. However, it has recently been emphasized that the pathways involved in regulating EMT are complex and diverse, which emphasizes the need for caution in linking stemness phenotypes in tumor cells (increased cell survival and drug resistance) to canonical pathways classically associated with EMT. See, for example, Yang et al., 2020, supra.

### SUMMARY

This document provides methods and materials involved in binding a molecule (e.g., an antibody, an antigen binding fragment, an antibody domain, a CAR, a cell engager, or an ADC) to a CSPG4 polypeptide. CSPG4 is a tumor cell surface oncoantigen. As described herein, CSPG4 is an independent risk factor for decreased survival of patients with EOC and can be used, for example, as a diagnostic biomarker in EOC. In addition, targeting cells that express CSPG can be used, for example, to limit recurrence and improve outcomes in patients with EOC or other CSPG4+ cancers. For example, as shown herein, CSPG4 promotes resistance to chemotherapy (e.g., cisplatin resistance), promotes tumor invasion and mesenchymal transition, and promotes the formation of multicelllular aggregates of tumor cells (spheroids). These spheroids are implicated in the development of peritoneal metastases, which are an important source of recurrence in ovarian cancer patients. Using CRISPR/Cas9 deletion of CSPG4 in multiple ovarian cancer cell lines, it was demonstrated that CSPG4 functions to promote several distinct phenotypic properties associated with malignant progression. These properties include increased tumor invasion, cisplatin resistance and enhanced formation of multicellular spheroids in vitro. CSPG4 also increased expression of multiple mesenchymal markers and promoted increased growth in vivo in a xenograft mouse model. Using immunohistochemical (IHC) analysis of a well-defined EOC patient cohort and mRNA expression level data from publicly available patient cohorts including The Cancer Genome Atlas (TCGA), it was determined that elevated levels of CSPG4 are linked to poor overall survival in multiple subtypes of EOC. These results functionally associate CSPG4 expression with multiple distinct cancer relevant phenotypes in EOC cells and link elevated CSPG4 expression to poor outcome in EOC patients. Furthermore, monoclonal anti-CSGP4 antibody inhibits CSPG4-stimulated ZEB1 expression, tumor cell invasion and promotes apoptosis of EOC cell in spheroids. The results described herein indicate that CSPG4 is a target for limiting recurrence and improving patient outcome.

In some embodiments, this document provides binders (e.g., antibodies, antigen binding fragments, antibody domains, CARs, cell engagers, or ADCs) that bind to a CSPG4 polypeptide and methods and materials for using one or more such binders to treat a mammal (e.g., a human) having cancer.

This document also provides cells (e.g., host cells) designed to express one or more binders (e.g., antibodies, antigen binding fragments, antibody domains, CARs, or cell engagers) having the ability to bind to a CSPG4 polypeptide and methods and materials for using such cells to treat cancer.

As described herein, binders (e.g., one or more antibodies, one or more antigen binding fragments, one or more antibody domains, one or more CARs, one or more cell engagers, and/or one or more ADCs) can be designed to have the ability to bind to a CSPG4 polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human CSPG4polypeptide as set forth in SEQ ID NO:33 (see, e.g., FIG. 1).

In some cases, two sets of three CDRs of an antigen binding fragment provided herein (e.g., SEQ ID NOs:1-3 and 9-11 or SEQ ID NOs:17-19 and 25-27) can be engineered into a CAR to create CAR⁺ cells (e.g., CAR⁺ T cells, CAR⁺ stem cells such as CAR⁺ induced pluripotent stem cells, or CAR⁺ natural killer (NK) cells) having the ability to target CSPG4⁺ cells (e.g., CSPG4⁺ tumor cells), can be engineered into an antibody structure that includes an Fc region to create antibodies having the ability to target CSPG4⁺ cells (e.g., CSPG4⁺ tumor cells) and induce antibody-dependent cell-mediated cytotoxicity (ADCC) against the target CSPG4⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target CSPG4⁺ cells (e.g., CSPG4⁺ tumor) and induce one or more immune responses (e.g., T cell immune responses and/or ADCC using a cell engager in the absence of an Fc-containing antibody) against the target CSPG4⁺ cells. It is noted that BiKE- and TriKE-mediated killing can be referred to ADCC even though it is not initiated by an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, and/or antibody domain-drug conjugates can be designed to include an appropriate binder provided herein to create the conjugate. Such conjugates can be used to deliver the drug payload to target cells such as cancer cells (e.g., CSPG4⁺ cancer cells).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer. For example, a mammal (e.g., a human) having cancer (e.g., a CSPG4⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer.

Binders (e.g., one or more antibodies, one or more antigen binding fragments, one or more antibody domains, one or more cell engagers, and/or one or more ADCs) also can be used to reduce tumor cell invasion, limit mesenchymal transition and/or inhibit spheroid formation.

As also described herein, cells (e.g., host cells) can be designed to express one or more binders (e.g., antibodies, antigen binding fragments, antibody domains, CARs, or cell engagers) having the ability to bind to a CSPG4 polypeptide. For example, cells such as T cells (e.g., CTLs), stem cells (e.g., induced pluripotent stem cells), or NK cells can be engineered to express one or more CARs having the ability to bind to a CSPG4 polypeptide. Such cells (e.g., CSPG4-specific CAR⁺ T cells or NK cells) can be used to treat cancer.

In one aspect, this document features an antibody that includes (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletion, or substitution), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can include the ability to bind to a human CSPG4 polypeptide (SEQ ID NO:33). In any of the embodiments, the antibody can be a monoclonal antibody or a scFv antibody.

In some embodiments, the antibody comprises the heavy chain variable domain or region of (i). The heavy chain variable domain or region can include an amino acid sequence having at least 90 percent identity to the amino acid sequence set forth in SEQ ID NO:8.

In some embodiments, the antibody comprises the light chain variable domain or region of (i). The light chain variable domain or region can include an amino acid sequence having at least 90 percent identity to the amino acid sequence set forth in SEQ ID NO:16.

In some embodiments, the antibody comprises the heavy chain variable domain or region of (ii). The heavy chain variable domain or region can include an amino acid sequence having at least 90 percent identity to the amino acid sequence set forth in SEQ ID NO:24.

In some embodiments, the antibody comprises the light chain variable domain or region of (ii). The light chain variable domain or region can include an amino acid sequence having at least 90 percent identity to the amino acid sequence set forth in SEQ ID NO:32.

This document also features an antigen binding fragment that includes (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletions or substitution), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can include the ability to bind to SEQ ID NO:33 or SEQ ID NO:34.

In some embodiments, the antigen binding fragment includes the heavy chain variable domain or region of (i). The heavy chain variable domain or region can include an amino acid sequence having at least 90 percent identity to the amino acid sequence set forth in SEQ ID NO:8.

In some embodiments, the antigen binding fragment includes the light chain variable domain or region of (i). The light chain variable domain or region can include an amino acid sequence having at least 90 percent identity to the amino acid sequence set forth in SEQ ID NO:16.

In some embodiments, the antigen binding fragment includes the heavy chain variable domain or region of (ii). The heavy chain variable domain or region can include an amino acid sequence having at least 90 percent identity to the amino acid sequence set forth in SEQ ID NO:24.

In some embodiments, the antigen binding fragment includes the light chain variable domain or region of (ii). The light chain variable domain or region can include an amino acid sequence having at least 90 percent identity to the amino acid sequence set forth in SEQ ID NO:32.

In any of the embodiments, the antigen binding fragment can be monoclonal. In any of the embodiments, the antigen binding fragment can be an Fab.

This document also features a nucleic acid that includes a nucleic acid sequence encoding at least part of an antibody or an antigen-binding fragment of any of the embodiments described herein and a host cell that includes such a nucleic acid. The nucleic acid sequence can encode the heavy chain variable domain or region of any of (i)-(ii). The nucleic acid sequence can encode the light chain variable domain or region of any of (i)-(ii). The nucleic acid can be a viral vector or a phagemid.

This document also features a chimeric antigen receptor that includes an antigen binding domain, a hinge, a transmembrane domain, and one or more signaling domains, wherein the antigen binding domain comprises an antibody or an antigen-binding fragment of any of the embodiments described herein. The antigen binding domain can include a scFv having the ability to bind to a CSPG4 polypeptide.

In another aspect, this document features a nucleic acid that includes a nucleic acid sequence encoding a chimeric antigen receptor of any of the embodiments described herein and a host cell that includes such a nucleic acid. The nucleic acid can be a viral vector or a phagemid.

This document also features a cell that includes a chimeric antigen receptor of any of the embodiments described herein. The cell can be a T cell, a stem cell, or an NK cell.

In another aspect, this document features a cell engager that includes a first antigen binding domain, a linker, and a second antigen binding domain, wherein the first antigen binding domain comprises an antibody or an antigen-binding fragment of any of the embodiments described herein. The first antigen binding domain can include a scFv having the ability to bind to a CSPG4 polypeptide. The first antigen binding domain can be an IgG having the ability to bind to a CSPG4 polypeptide. The second antigen binding domain can bind to a polypeptide expressed on the surface of T cells (e.g., a CD3 polypeptide) or NK cells (e.g., a CD16a polypeptide). The cell engager can include a third antigen binding domain, e.g., a third antigen binding domain that bind to a polypeptide expressed on the surface of NK cells such as a CD16a polypeptide.

In another aspect, this document features a nucleic acid that includes a nucleic acid sequence encoding a cell engager of any of the embodiments described herein and a host cell that includes such a nucleic acid. The nucleic acid can be a viral vector or a phagemid.

This document also features a host cell that expresses a chimeric antigen receptor or a cell engager described herein. The host cell can be a T cell, stem cell, or NK cell.

In another aspect, this document features an antibody-drug conjugate (ADC) that includes an antigen binding domain covalently linked to a drug, wherein the antigen binding domain comprises an antibody or an antigen binding fragment of any of the embodiments described herein. The antigen binding domain can include a scFv having the ability to bind to a CSPG4 polypeptide or an IgG having the ability to bind to a CSPG4 polypeptide. The drug can be selected from the group consisting of calicheamicin, monomethyl auristatin E (MMAE), emtansine (DM1), and an exatecan derivative (Dxd).

This document also features a composition comprising an antibody or an antigen binding fragment described herein, a cell engager described herein, a cell described herein, or an ADC described herein. The composition also can include a checkpoint inhibitor (e.g., a checkpoint inhibitor selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab).

A method of treating a mammal (e.g., a human) having cancer also is featured. The method includes administering, to the mammal (e.g., a human), a composition described herein. The cancer can be a CSPG4+ cancer such as CSPG4+ ovarian cancer. The number of cancer cells within the mammal (e.g., human) can be reduced following the administering step.

This document also features a method for binding a binding molecule to a CSPG4 polypeptide. The method includes contacting the CSPG4 polypeptide with an antibody or an antigen binding fragment described herein or contacting the CSPG4 polypeptide with a chimeric antigen receptor, a cell engager, or an ADC described herein. The contacting can be performed in vitro or in vivo. For example, the contacting can be performed within a mammal (e.g., human) by administering the antibody or the antigen binding fragment to the mammal (e.g., human). For example, the contacting can be performed within a mammal (e.g., human) by administering the chimeric antigen receptor, cell engager, or ADC to the mammal (e.g., human).

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1D. Antibody 7H5A2 targets CSPG4. FIG. 1A) Amino acid sequence of recombinant protein target used to generate antibody 7H5A2 (SEQ ID NO:34). Black text denotes CSPG4 region (SEQ ID NO:33), the lighter gray text corresponds to human FC sequence for protein purification. FIGS. 1A and 1B) Western blot with CSPG4 antibodies 763.74 (FIG. 1B) and 7H5A2 (FIG. 1C). Cells are harvested in lysis buffer and 40 μg protein was loaded in each well from the indicated mock and CSPG4-CRISPR cell lines. Alpha-tubulin is included as a loading control. FIG. 1D) Immunofluorescence staining of HEY parent cells with antibody 7H5A2. Red bar=10 μm. Magnification 600×.

FIGS. 2A-2C. CSPG4 protein expression characterized in 126 patient ovarian cancer cohort. FIG. 2A) Representative images from IHC staining for CSPG4 in the ovarian cancer patient cohort. Staining intensity is scored on a scale from 0 to 3 [0 (negative), 1 (weak), 2 (moderate) and 3 (strong)]. Tumor tissue staining indicated by red arrows, stromal staining indicated by black arrow. The fraction of CSPG4 staining is scored from 0 to 4, reflecting the percentage of positively stained tumor cells in the sample [0 (0%), 1 (1-25%), 2 (25-50%), 3 (50-75%) and 4 (75-100%)]. The intensity and fraction positive scores were added together to generate the total score (TS). TS≥4 was considered high expression of CSPG4, while TS≤3 was considered low expression. A TS of zero was considered negative. Red bar=20 μm. FIGS. 2B and 2C) Kaplan-Meier curves over 40 months for censored data from this 126-patient ovarian cancer cohort. FIG. 2B) CSPG4 high expression (green line; 24/29) vs. low CSPG4 blue line (33/48) correlates with reduced progression free survival (PFS: 22.615±1.754 vs 16.559±1.940, X2=4.316, P=0.038). FIG. 2C) High CSPG4 expression (green line 19/29) vs low CSPG4 (blue line 22/48) correlates with reduced overall survival overall survival (OS: 31.027±1.353 vs 24.046±2.177, X2=7.366, P=0.007).

FIGS. 3A-3B. CRISPR knockout of CSPG4 in A2780 ovarian carcinoma cells results in reduced tumor growth in vivo. NSG mice were injected I.P. with 2.0×10⁵ luc+A2780 Mock or A2780 CSPG4-CRISPR knockout cells. FIG. 3A shows tumor growth was monitored by bioluminescent imaging (BLI) on day 6, 13, and 27. Color scale bar indicates photon/s/cm²/sr. FIG. 3B shows the quantification of tumor burden based on BLI total flux (photons/sec). Data are shown as mean±SD. ****p<0.0001 by ordinary two-way ANOVA with Sidak's multiple comparison test. Mock: n=4, CRISPR, n=5, Negative control, n=5.

FIGS. 4A-4C. CSPG4 expression in parent and CRISPR cell lines by flow cytometry. ES-2 (FIG. 4A) and HEY (FIG. 4B) parent and CRISPR knockout cells were stained with either normal mouse IgG1 or anti-CSPG4 antibody 763.74. FIG. 4C) A2780 parent and CRISPR knockout cells were stained with either normal mouse IgG2a or anti-CSPG4 antibody 9.2.27.

FIGS. 5A-5G. CSPG4 knockout results in significant loss of invasive capacity and cisplatin resistance in multiple ovarian tumor cell lines. Invasion assays using control (Mock) and CSPG4 knockout (CRISPR) HEY cells (FIG. 5A) or A2780 cells (FIG. 5B). Bars represent the total number of invading cells from five random fields/well from triplicate wells, +/−S.D., from three replicate experiments, n=6. P values determined by student's t-test with Welch's correction. FIG. 5C shows a Western blot for CSPG4 in ES2 cell lines. Lanes: 1—ES-2 Parent, 2—ES-2 Mock, 3—ES-2 CSPG4 knockout, 4—ES-2 CSPG4 Rescue. FIG. 5D shows an invasion assay using indicated ES-2 cell lines. Invasive capacity is rescued in the ES-2 CRISPR knockout line with CSPG4 re-expression (Rescue). P values determined by student's t-test with Welch's correction. FIGS. 5E-G show the cell viability of mock and knockout (CRISPR) A2780 cells (FIG. 5E), mock and knockout HEY cells (FIG. 5F), and mock, knockout and rescue ES-2 cells (FIG. 5G) treated with increasing concentrations of cisplatin. Dose response curves were plotted as the percent of MTS staining vs. untreated cells for each cell line +/−s.e.m. from three replicate experiments (n=9). The phenotype was rescued by re-expression of CSPG4 in the ES-2 CRISPR cells (ES-2 Rescue, in FIG. 5G).

FIG. 6. All enriched GO terms identified in differentially expressed genes between the ES-2 CRISPR knockout cells vs the mean expression level in the ES-2 parental and mock cell lines. There were a total of 388 enriched GO terms identified. Highly significant GO terms (adjusted p-value <0.005) of particular functional interest are highlighted.

FIGS. 7A-7E. Knockout of CSPG4 decreased anchorage-independent growth and spheroid formation. FIG. 7A) ES-2 parent, Mock, CRISPR and stable CSPG4-rescue cells were grown in a soft agar colony formation assay for seven days. Bars represent the total number colonies counted from five random fields/well from replicate wells, +/−S.E., n=6. P values determined by student's t-test with Welch's correction. FIGS. 7B and 7C) CSPG4-CRISPR cells form fewer/smaller spheroids when plated in methylcellulose media. FIG. 7B) Representative images showing larger and more spheroids from Mock and small and few spheroids from CSPG4-CRISPR cultures for three EOC cell lines. Bar=200 μm. FIG. 7C) Spheroid counts from five random fields/well from three replicate experiments+/−S.D. *p<0.001 by students t-test with Welch's correction. Spheroids were quantified 7 days post plating. n=3. FIG. 7D) Cells (Mock,M; CRISPR,C; CRISPR rescued with CSPG4 re-expression,R) were cultured in 1.0% methylcellulose/complete media for 7 days, harvested and lysates analyzed by western blot for FAK expression/phosphorylation. FIG. 7E) Cell growth in spheroids. Cells were plated in 1% methylcellulose for 7 days in duplicate wells, collected from methylcellulose culture and re-plated overnight in normal growth media on tissue culture plates. The following day all cells were collected from each well and counted. P values determined by student's t-test with Welch's correction, comparing live cell numbers (determined by trypan blue exclusion) between Mock and CRISPR cells for each cell line, n=4.

FIGS. 8A-8B. CSPG4 expression is associated with epithelial-to-mesenchymal plasticity. FIG. 8A) Gene set enrichment analysis (GSEA) of RNA-seq data comparing parent, mock and CRISPR knockout ES-2 cell lines. The ES-2 CRISPR cell line shows an enrichment in the expression of EMT associated genes when compared to the mock and parental cell lines. The Normalized Enrichment Score is 2.190. FIG. 8B) Ovarian cancer TCGA cohort analyzed for mean CSPG4 expression and EMT signature score.

FIGS. 9A-9J. CSPG4 expression is associated with epithelial-to-mesenchymal plasticity, mediated by CSPG4 associated changes in ZEB1 expression. FIG. 9A) The indicated cell lines both Mock(M) and CRISPR(C) were cultured separately in spheroid formation assays for 7 days, collected and analyzed by western blot for various EMT markers. FIG. 9B) Western blot for Zebl and CSPG4 expression in EOC cell lines treated with siRNA for ZEB1 or control for 48 hours. FIG. 9C) Western blot for pFAK, FAK and ZEB1 in HEY cells treated with either DMSO (control) or FAK inhibitor PND-1186 at the indicated concentrations in normal growth medium for 24 hours. FIG. 9D) Spheroid formation in methylcellulose of EOC cell lines treated with siRNA to ZEB1 or control for 7 days. Bars represent spheroid counts from five random fields/well from triplicate wells+/−S.D. *p<0.02 by students t-test with Welch's correction. n=3 from 3 individual experiments. FIG. 9E) Invasion assay of EOC cells treated with ZEB1 siRNA or control siRNA (methods). *p<0.002 by student's t-test with Welch's correction. n=3 from 3 individual experiments. FIGS. 9F and 9G) Cells were transfected with control or ZEB1 siRNA overnight and plated in the cisplatin cytotoxicity assay the following day. MTS data was collected after 4 days of cisplatin treatment. Untransfected CSPG4-CRISPR cells for A2780 (FIG. 9F) and HEY (FIG. 9G) were included as a control. Dose response curves were plotted as the percent of MTS staining vs. untreated cells for each group. Combined data from three independent experiments are shown (n=9). FIGS. 9H-9J) Kaplan-Meier curves of ovarian cancer patients in a combined cohort of TCGA and 14 GEO datasets demonstrate that tumors that express CSPG4 (FIG. 9H), ZEB1 (FIG. 9I), and the mean combined expression of both (FIG. 9J) are associated with decreased 5-year survival (red lines) when compared to data from tumors that are negative for these markers (black lines)

FIGS. 10A-10D. Anti-CSPG4 antibody 7H5A2 inhibits Zeb1 expression and FAK activation, cell invasion and spheroid formation. FIG. 10A) Western blot for pFAK, FAK, and ZEB1 in EOC cells plated in 1% methylcellulose in the presence of 50 μg/ml normal mouse IgG1 (nmIgG1) or anti-CSPG4 antibody 7H5A2 cultured in suspension for 24 hours. FIG. 10B) Invasion assay using HEY cells pre-incubated with control (nm IgG1) or anti-CSPG4 (763.74 and 7H5A2) antibodies at 50 μg/ml for 2 hours and then plated in invasion chambers in the presence of the antibody in the top chamber only for 24 hours. Bars represent the total number of invading cells counted in five random fields/well from duplicate wells, +/−S.D. P values determined by student's t-test with Welch's correction. n=6 from 3 separate experiments. FIG. 10C) Cell growth assay using EOC cells plated in the presence of the indicated antibodies at 50 μg/ml in 1% methylcellulose for 7 days (HEY) or 14 days (A2780 and ES-2). Cells were collected from methylcellulose culture at those time points, and re-plated overnight in normal growth media in tissue culture plates. The following day all cells were collected from each well and counted. P values determined by student's t-test with Welch's correction, comparing live cell numbers (determined by trypan blue exclusion) from each condition, n=4. FIG. 10D) Cells were pre-treated with normal mouse IgG1 or antibody 7H5A2 (50 μg/ml) for 1 hour and then plated in 1% methylcellulose supplemented with 50 μg/ml of the indicated antibody. Spheroids were harvested after 72 hours and assayed for caspase-3 activation by western blot. Apoptosis of spheroids

FIGS. 11A-11C. FIG. 11A) HEY Mock and CRISPR cells were treated with the indicated anti-CSPG4 monoclonal antibodies or normal mouse IgG1 (nmIgG1). The indicated NK92 cell lines were added at a 1:1 effector:target ratio and ADCC determined at 4 hrs using the DELFIA EuTDA cytotoxicity assay following the manufacturer's protocol. Bars represent the percent specific cell lysis for triplicate samples+/−s.e.m. *p<0.05, ****p<0.0001 vs. nmIgGl control by two-way ANOVA with Dunnett's multiple comparisons test. ADCC of spheroids. FIG. 111B) A repeat of the experiment of FIG. 11A. **p=0.0088, ***p<0.0009 by 2 way ANOVA with Dunnett's multiple comparisons test compared to nmIgGl control; ** and ***p<0.0009 vs no Ab control. ADCC of spheroids. FIG. 11C) OVCAR8 Mock and CRISPR cells were treated with the indicated anti-CSPG4 monoclonal antibodies or normal mouse IgG1 (nmIgGl). The indicated NK92 cell lines were added at a 1:1 effector:target ratio and ADCC determined at 4 hrs using the DELFIA EuTDA cytotoxicity assay following the manufacturer's protocol. Bars represent the percent specific cell lysis for triplicate samples. **p=0.0038, by 2 way ANOVA with Dunnett's multiple comparisons test compared to nmIgG1 control; **p=0.0315 vs no Ab control. ADCC of high grade serous cell spheroids

FIG. 12. CSPG4 expression in ovarian cancer cell lines. The indicated cell lines were assayed for CSPG4 expression by western blot with anti-CSPG4 antibody 9.2.27 (Millipore). Western blots were probed for tubulin as a loading control. Both SKOV-3 and OVCAR-5 cell lines were negative for CSPG4, while the other cell lines expressed varying amounts of CSPG4 protein. The OVCAR-8 western blot images are from a separate western blot from a separate experiment than the other cell lines.

FIGS. 13A-13B. CSPG4 TriKEs enhance NK cell mediated killing of CSPG4-positive ovarian cancer tumor spheroids. GFP-expressing OVCAR-8 cells (FIG. 13A) or CSPG4-negative SKOV3 ovarian cancer cells (FIG. 13B), were cultured in ultra-low attachment of 96 well-round bottom plate (20,000 cells/well) for 2 days for spheroid formation. Enriched NK cells were added to wells at an effector:target ratio of 2:1 (40,000/well) with no treatment, CSPG4 TriKE 8G5A6 (30 nM), CSPG4 TriKE 7H5A2 (30 nM), or IL-15 (3 nM). Images were taken of each well upon NK addition and at 2-hour increments over 4 days using the IncuCyte SX5-Live Cell Analysis Platform. Graphs show the mean fluorescence intensity at each time point as a percent of untreated tumor spheroids (normalized to time 0) for each condition from three technical replicates.

FIG. 14. A schematic of an exemplary BiTE designed using CDR1, CDR2, and CDR3 of a heavy chain provided herein and CDR1, CDR2, and CDR3 of a light chain provided herein in an Ig format (e.g., an IgG1 format). A humanized anti-CD3 scFv (e.g., an gOKT3-7 scFv set forth in U.S. Pat. No. 6,750,325) can be linked to the C-terminus of the light chain via a linker (e.g., a (SGGGG)₃₋₅ (SEQ ID NO:35) linker).

### DETAILED DESCRIPTION

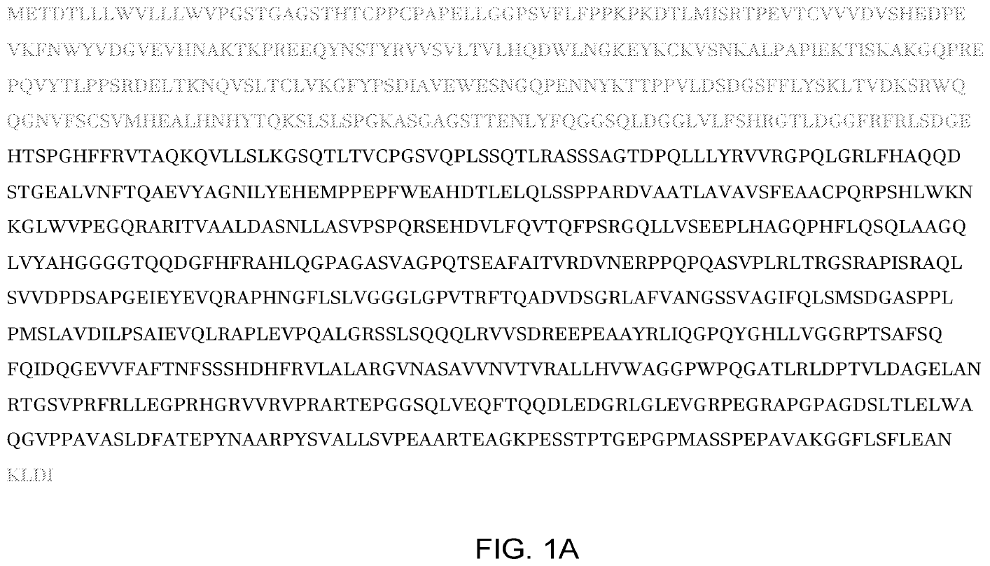

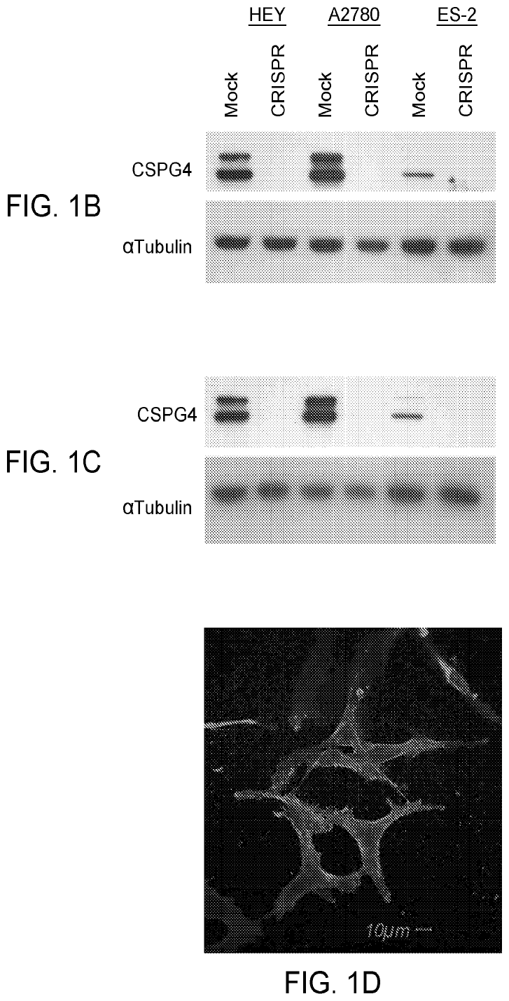

This document provides binders (e.g., antibodies, antigen binding fragments, antibody domains, CARs, cell engagers, and ADCs) that bind (e.g., specifically bind) to a CSPG4 polypeptide (e.g., a human CSPG4 polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the CSPG4 amino acid set forth in FIG. 1 (black text in FIG. 1, SEQ ID NO:33). The binders described herein were generated against the juxtamembrane (D3) region of a CSPG4 polypeptide, a region which has been previously linked to regulating β1 integrin and cell motility. Targeting a CSPG4 polypeptide with the binders described herein can inhibit ZEB1 expression and thus can limit CSPG4 mediated epithelial to mesenchymal shift. In some embodiments, the binders described herein can block invasion and promote apoptosis of CSPG4-positive spheroids. This indicates that the anti-CSPG4 binders provided herein have additional functions on cell survival, and can be used to target spheroids containing CSPG4^{-ve} EOC tumor cells. In some cases, binders generated against the juxtamembrane domain can be used as therapies and can be used to inhibit tumor expansion and metastasis in vivo.

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Farber-Schwarz, Methods Mol. Biol., 907:713-27 (2012); and Brinkmann and Kontermann, MAbs., 9(2):182-212 (2017)). The two variable regions can be connected by a polypeptide linker (e.g., a polypeptide linker having five to ten residues in length). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length). See, also, Chen et al., Adv. Drug Deliv. Rev., 65(10):1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains.

An antibody provided herein can include the CDRs as described herein (e.g., as described in Table 1) and can be configured to be a murine antibody, a humanized antibody, or a chimeric antibody. In some cases, an antibody provided herein can include the CDRs as described herein (e.g., as described in Table 1) and can be a monoclonal antibody. In some cases, an antibody provided herein can include the CDRs as described herein (e.g., as described in Table 1) and can be configured as a scFv antibody.

The term “antigen binding fragment” as used herein refers to a fragment of an antibody (e.g., a fragment of a humanized antibody, a fragment of a murine antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 1) and can be configured to be a murine antigen binding fragment, a humanized antigen binding fragment, or a chimeric antigen binding fragment. In some cases, an antigen binding fragment provided herein can include the CDRs as described herein (e.g., as described in Table 1) and can be a monoclonal antigen binding fragment. In some cases, an antigen binding fragment provided herein can include the CDRs as described herein (e.g., as described in Table 1) and can be configured as a Fab antibody. In some cases, a Fab antibody can include a partial hinge sequence for disulfide bonding between heavy and light chains of the Fab.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 1) and can be a murine antibody domain, a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 1) and can be a monoclonal antibody domain. In some cases, an antibody domain provided herein can include the CDRs as described herein (e.g., as described in Table 1) and can be engineered as a single VH domain or a single VL domain.

An anti-CSPG4 antibody, anti-CSPG4 antigen binding fragment, or anti-CSPG4 antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-CSPG4 antibody) can be a scFv antibody. In some cases, an antigen binding fragment provided herein (e.g., an anti-CSPG4 antibody fragment) can be a Fab. In some cases, an antibody provided herein (e.g., an anti-CSPG4 antibody) can be a fully intact antibody. In some cases, an antibody domain provided herein (e.g., an anti-CSPG4 antibody domain) can be a VH domain.

The term “chimeric antigen receptor” as used herein refers to a chimeric polypeptide that is designed to include an optional signal peptide, an antigen binding domain, an optional hinge, a transmembrane domain, and one or more intracellular signaling domains. As described herein, the antigen binding domain of a CAR provided herein can be designed to bind to a CSPG4 polypeptide (e.g., a human CSPG4 polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a CSPG4 polypeptide (e.g., a human CSPG4 polypeptide). In some examples, a CAR provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11 or SEQ ID NOs:17-19 and 25-27). In some cases, an antigen binding domain of a CAR targeting a CSPG4 polypeptide can be designed to include a VH domain described herein or a scFv antibody described herein.

In some cases, a CAR provided herein can be designed to include a signal peptide. Any appropriate signal peptide can be used to design a CAR described herein. Examples of signal peptide that can be used to make a CAR described herein include without limitation, a tPA signal peptide, BiP signal peptide, or CD8a signal peptide.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence (e.g., (SGGGG)₂₋₅ (SEQ ID NO:36)) can be used as a hinge to make a CAR described herein.

A CAR provided herein can be designed to include any appropriate transmembrane domain. For example, the transmembrane domain of a CAR provided herein can be, without limitation, a CD3ζ transmembrane domain, a CD4 transmembrane domain, a CD8a transmembrane domain, a CD28 transmembrane domain, and a 4-1BB transmembrane domain.

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. See, e.g., Feins, et al., Am J Hematol., 94(S1):S3-S9 (2019).

In some cases, a CAR targeting a CSPG4 polypeptide can be designed to include an scFv having a heavy chain variable domain comprising SEQ ID NO:1, SEQ ID NO:2, and SEQ ID NO:3, followed by a linker such as (SGGGG)₂₋₅ (SEQ ID NO:36), followed by a light chain variable domain comprising SEQ ID NO:9, SEQ ID NO:10, and SEQ ID NO:11, followed by a hinge such as a hinge/linker (e.g., an IgG4-derived hinge, a CD8α hinge, or a linker plus IgG4-derived hinge), followed by a transmembrane domain (e.g., a human CD28 transmembrane domain or a CD8α transmembrane domain), followed by one or more intracellular signaling domains.

In some cases, a CAR targeting a CSPG4 polypeptide can be designed to include an scFv having a heavy chain variable domain comprising SEQ ID NO:8, followed by a linker such as (SGGGG)₂₋₅ (SEQ ID NO:36), followed by a light chain variable domain comprising SEQ ID NO:16, followed by a hinge such as a hinge/linker (e.g., an IgG4-derived hinge, a CD8α hinge, or a linker plus IgG4-derived hinge), followed by a transmembrane domain, followed by one or more intracellular signaling domains such as one or more intracellular signaling domain.

In some cases, a CAR targeting a CSPG4 polypeptide can be designed to include an scFv having a light chain variable domain comprising SEQ ID NO:9, SEQ ID NO:10, and SEQ ID NO:11, followed by a linker such as (SGGGG)₂₋₅ (SEQ ID NO:36), followed by a heavy chain variable domain comprising SEQ ID NO:1, SEQ ID NO:2, and SEQ ID NO:3, followed by a hinge such as a hinge/linker (e.g., an IgG4-derived hinge, a CD8α hinge, or a linker plus IgG4-derived hinge), followed by a transmembrane domain, followed by one or more intracellular signaling domains.

In some cases, a CAR targeting a CSPG4 polypeptide can be designed to include an scFv having a light chain variable domain comprising SEQ ID NO:16, followed by a linker such as (SGGGG)₂₋₅ (SEQ ID NO:36), followed by a heavy chain variable domain comprising SEQ ID NO:8, followed by a hinge such as a hinge/linker (e.g., an IgG4-derived hinge, a CD8α hinge, or a linker plus IgG4-derived hinge), followed by a transmembrane domain (e.g., a human CD28 transmembrane domain or a CD8α transmembrane domain), followed by one or more intracellular signaling domains.

In some cases, a CAR targeting a CSPG4 polypeptide can be designed to include an scFv having a heavy chain variable domain comprising SEQ ID NO:17, SEQ ID NO:18, and SEQ ID NO:19, followed by a linker (SGGGG)₂₋₅ (SEQ ID NO:36), followed by a light chain variable domain comprising SEQ ID NO:25, SEQ ID NO:26, and SEQ ID NO:27, followed by a hinge (e.g., an IgG4-derived hinge, a CD8α hinge, or a linker plus IgG4-derived hinge), followed by a transmembrane domain (e.g., a human CD28 transmembrane domain or a CD8α transmembrane domain), followed by one or more intracellular signaling domains.

In some cases, a CAR targeting a CSPG4 polypeptide can be designed to include an scFv having a heavy chain variable domain comprising SEQ ID NO:24, followed by a linker such as (SGGGG)₂₋₅ (SEQ ID NO:36), followed by a light chain variable domain comprising SEQ ID NO:32, followed by a hinge such as a hinge/linker (e.g., an IgG4-derived hinge, a CD8α hinge, or a linker plus IgG4-derived hinge), followed by a transmembrane domain (e.g., a human CD28 transmembrane domain or a CD8α transmembrane domain), followed by one or more intracellular signaling domains.

In some cases, a CAR targeting a CSPG4 polypeptide can be designed to include an scFv having a light chain variable domain comprising SEQ ID NO:25, SEQ ID NO:26, and SEQ ID NO:27, followed by a linker such (SGGGG)₂₋₅ (SEQ ID NO:36), followed by a heavy chain variable domain comprising SEQ ID NO:17, SEQ ID NO:18, and SEQ ID NO:19, followed by a hinge such as a hinge/linker (e.g., an IgG4-derived hinge, a CD8α hinge, or a linker plus IgG4-derived hinge), followed by a transmembrane domain, followed by one or more intracellular signaling domains.

In some cases, a CAR targeting a CSPG4 polypeptide can be designed to include an scFv having a light chain variable domain comprising SEQ ID NO:32, followed by a linker such as (SGGGG)₂₋₅ (SEQ ID NO:36), followed by a heavy chain variable domain comprising SEQ ID NO:24, followed by a hinge such as a hinge/linker (e.g., an IgG4-derived hinge, a CD8α hinge, or a linker plus IgG4-derived hinge), followed by a transmembrane domain (e.g., a human CD28 transmembrane domain or a CD8α transmembrane domain), followed by one or more intracellular signaling domains.

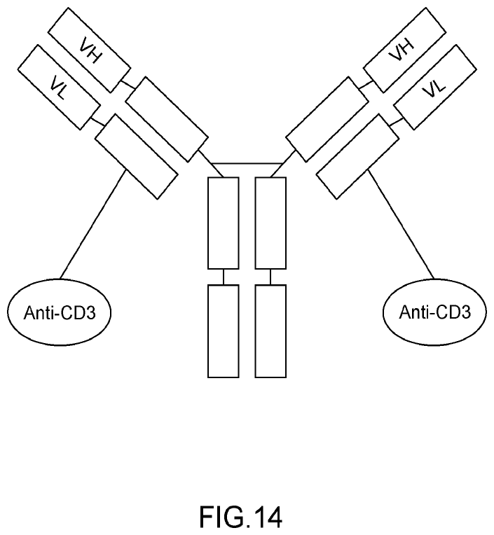

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, and TriKEs. In general, a cell engager provided herein can be designed to include at least one antigen binding domain having the ability to bind to a CSPG4 polypeptide (e.g., a human CSPG4 polypeptide) and at least one antigen binding domain having the ability to bind to an antigen expressed on the surface of a cell (e.g., a T cell or an NK cell). In some cases, a cell engager described herein can link a CSPG4⁺ cell (e.g., a CSPG4⁺ cancer cell) to another cell (e.g., a T cell or an NK cell) via the two or more antigen binding domains of the cell engager. An example of a cell engager structure of cell engagers provided herein includes, without limitation, the structure set forth in FIG. 14. In some cases, the anti-CD3 scFv depicted in FIG. 14 can be replaced with a different antigen binding domain having the ability to bind to an antigen expressed on the surface of a cell (e.g., a T cell or an NK cell).

When a cell engager includes an antigen binding domain having the ability to bind to a CSPG4 polypeptide (e.g., a human CSPG4 polypeptide) and one or more other antigen binding domains (e.g., two, three, or four other antigen binding domains), each of those other antigen binding domains can bind to different antigens expressed on the surface of different cell types or can bind to different antigens expressed on the surface of the same cell type. In some embodiments, a TriKE can be designed to have a first antigen binding domain having the ability to bind to a CSPG4 polypeptide (e.g., a human CSPG4 polypeptide), a second antigen binding domain having the ability to bind to a first antigen expressed on the surface of an NK cell (e.g., a CD16 polypeptide such as a CD16a polypeptide), and a third moiety such as IL-15 that promotes NK cell specific proliferation. See, e.g., Arvindam, et al., Leukemia, 35(6):1586-1596 (2021). In some embodiments, the third moiety is a third antigen binding domain having the ability to bind to a second antigen expressed on the surface of an NK cell (e.g., a CD16, NKG2D, NKG2C, NKp46, NKp30, or NKp44).

As described herein, at least one antigen binding domain of a cell engager provided herein can be designed to bind to a CSPG4 polypeptide (e.g., a human CSPG4 polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to a CSPG4 polypeptide (e.g., a human CSPG4 polypeptide). In some examples, a cell engager provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11 or SEQ ID NOs:17-19 and 25-27). In some cases, an antigen binding domain of a cell engager targeting a CSPG4 polypeptide can be designed to include a VH domain described herein or a scFv/Fab antibody described herein. In some cases, an antigen binding domain of a CAR described herein that has the ability to bind to a CSPG4 polypeptide (e.g., a human CSPG4 polypeptide) can be used as an antigen binding domain of a cell engager that targets CSPG4⁺ cells.

As described herein, a cell engager can be designed to include at least one antigen binding domain having the ability to bind to a CSPG4 polypeptide (e.g., a human CSPG4 polypeptide) and at least one other antigen binding domain. The at least one other antigen binding domain can have the ability to bind to any appropriate antigen expressed on the surface of a cell. For example, when designing a cell engager such as a BiTE to link a CSPG4⁺ cell and a T cell, the cell engager can include an antigen binding domain having the ability to bind to a CSPG4 polypeptide (e.g., a human CSPG4 polypeptide) and an antigen binding domain having the ability to bind to a polypeptide expressed on the surface of a T cell. Examples example of polypeptides expressed on the surface of a T cell that can be targeted by an antigen binding domain of a cell engager provided herein include, without limitation, CD3 polypeptides, NKG2D, TCR, and CD28. Examples of antigen binding domains having the ability to bind to a polypeptide expressed on the surface of a T cell that can be used to make a cell engager provided herein (e.g., a BiTE) include, without limitation, anti-CD3 scFvs and anti-CD3 VH domains, CD28, TCR, and NKG2D. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

When designing a cell engager such as a BiKE or a TriKE to link a CSPG4⁺ cell and an NK cell, the cell engager can include an antigen binding domain having the ability to bind to a CSPG4 polypeptide (e.g., a human CSPG4 polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16 polypeptides (e.g., CD16a polypeptides), CD16, NKG2D, NKG2C, NKp46, NKp30, and NKp44. Examples of antigen binding domains having the ability to bind to a polypeptide expressed on the surface of an NK cell that can be used to make a cell engager provided herein (e.g., a BiKE or TriKE) include, without limitation, anti-CD16a scFvs and anti-CD16a VH domains. Additional examples of amino acid sequences that can be used as antigen binding domains having the ability to bind to a polypeptide expressed on the surface of an NK cell (e.g., CD16, NKG2D, NKG2C, NKp46, NKp30, or NKp44) are described in McCall et al. (Mol. Immunol., 36(7):433-445 (1999); see, e.g., anti-CD16 scFv sequences); International Patent Application Publication No. PCT/US2017/048721 (see, e.g., the CDRs and sequence listing for anti-CD16a binding domains).

In some cases, a cell engager provided herein can be designed to include a linker located between each antigen binding domain. Any appropriate linker can be used to design a cell engager provided herein, including, without limitation, (SGGGG)₃₋₅ (SEQ ID NO:35). A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a (SGGGG)₃₋₅ (SEQ ID NO:35) linker. In some cases, a hinge of a CAR described herein can be used as a linker to make a cell engager described herein.

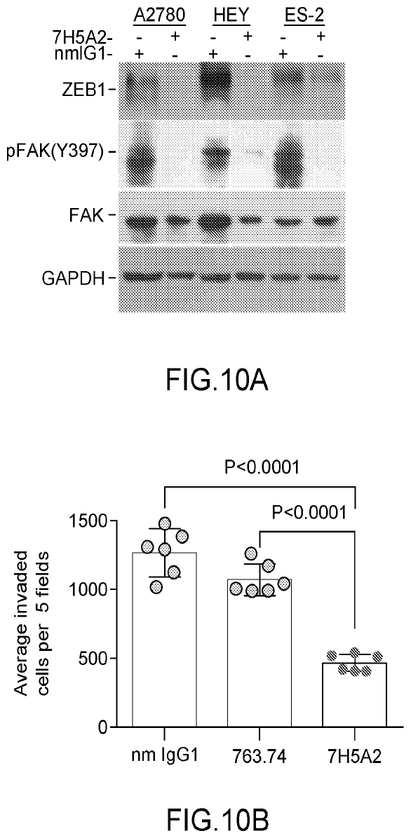

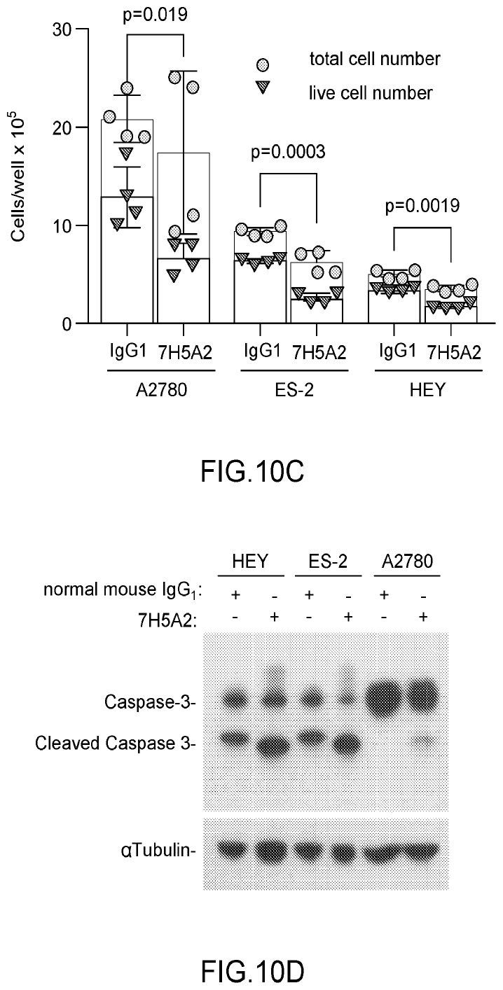

In some cases, a cell engager (e.g., a BiTE) targeting a CSPG4 polypeptide can be designed to include an scFv having a heavy chain variable domain comprising SEQ ID NO:1, SEQ ID NO:2, and SEQ ID NO:3, followed by a linker such as a linker set forth in FIG. 10, followed by a light chain variable domain comprising SEQ ID NO:9, SEQ ID NO:10, and SEQ ID NO:11, followed by a linker (SGGGG)₃₋₅ (SEQ ID NO:35), followed by an antigen binding domain having the ability to bind to a polypeptide expressed on the surface of a T cell (e.g., an anti-human CD3 scFv).

In some cases, a cell engager (e.g., a BiTE) targeting a CSPG4 polypeptide can be designed to include an scFv having a light chain variable domain comprising SEQ ID NO:9, SEQ ID NO:10, and SEQ ID NO:11, followed by a linker such as (SGGGG)₃₋₅ (SEQ ID NO:35), followed by a heavy chain variable domain comprising SEQ ID NO:1, SEQ ID NO:2, and SEQ ID NO:3, followed by a linker such as (SGGGG)₃₋₅ (SEQ ID NO:35), followed by an antigen binding domain having the ability to bind to a polypeptide expressed on the surface of a T cell (e.g., an anti-human CD3 scFv).

In some cases, a cell engager (e.g., a BiTE) targeting a CSPG4 polypeptide can be designed to include an scFv having a heavy chain variable domain comprising SEQ ID NO:8, followed by a linker such as (SGGGG)₃₋₅ (SEQ ID NO:35), followed by a light chain variable domain comprising SEQ ID NO:16, followed by a linker such as a hinge/linker, followed by an antigen binding domain having the ability to bind to a polypeptide expressed on the surface of a T cell (e.g., an anti-human CD3 scFv).

In some cases, a cell engager (e.g., a BiTE) targeting a CSPG4 polypeptide can be designed to include an scFv having a light chain variable domain comprising SEQ ID NO:16, followed by a linker such as (SGGGG)₃₋₅ (SEQ ID NO:35), followed by a heavy chain variable domain comprising SEQ ID NO:8, followed by a linker such as a hinge/linker, followed by an antigen binding domain having the ability to bind to a polypeptide expressed on the surface of a T cell (e.g., an anti-human CD3 scFv).

In some cases, a cell engager (e.g., a BiKE or a TriKE) targeting a CSPG4 polypeptide can be designed to include an scFv having a heavy chain variable domain comprising SEQ ID NO:1, SEQ ID NO:2, and SEQ ID NO:3, followed by a linker such as (SGGGG)₃₋₅ (SEQ ID NO:35), followed by a light chain variable domain comprising SEQ ID NO:9, SEQ ID NO:10, and SEQ ID NO:11, followed by a linker such as a hinge/linker, followed by one or more antigen binding domains having the ability to bind to a polypeptide expressed on the surface of an NK cell (e.g., an anti-human CD16a scFv for a BiKE or an anti-human CD16a scFv and an anti-human CD16, NKG2D, NKG2C, NKp46, NKp30, or NKp44 scFv for a TriKE).

In some cases, a cell engager (e.g., a BiKE or a TriKE) targeting a CSPG4 polypeptide can be designed to include an scFv having a light chain variable domain comprising SEQ ID NO:9, SEQ ID NO:10, and SEQ ID NO:11, followed by a linker such as (SGGGG)₃₋₅ (SEQ ID NO:35), followed by a heavy chain variable domain comprising SEQ ID NO:1, SEQ ID NO:2, and SEQ ID NO:3, followed by a linker such as a hinge/linker, followed by one or more antigen binding domains having the ability to bind to a polypeptide expressed on the surface of an NK cell (e.g., an anti-human CD16a scFv for a BiKE or an anti-human CD16a scFv and an anti-human CD16, NKG2D, NKG2C, NKp46, NKp30, or NKp44 scFv for a TriKE).

In some cases, a cell engager (e.g., a BiKE or a TriKE) targeting a CSPG4 polypeptide can be designed to include an scFv having a heavy chain variable domain comprising SEQ ID NO:8, followed by a linker such as (SGGGG)₃₋₅ (SEQ ID NO:35), followed by a light chain variable domain comprising SEQ ID NO:16, followed by a linker such as a hinge/linker, followed by one or more antigen binding domains having the ability to bind to a polypeptide expressed on the surface of an NK cell (e.g., an anti-human CD16a scFv for a BiKE or an anti-human CD16a scFv and an anti-human CD16, NKG2D, NKG2C, NKp46, NKp30, or NKp44 scFv for a TriKE).

In some cases, a cell engager (e.g., a BiKE or a TriKE) targeting a CSPG4 polypeptide can be designed to include an scFv having a light chain variable domain comprising SEQ ID NO:16, followed by a linker such as (SGGGG)₃₋₅ (SEQ ID NO:35), followed by a heavy chain variable domain comprising SEQ ID NO:8, followed by a linker such as a hinge/linker, followed by one or more antigen binding domains having the ability to bind to a polypeptide expressed on the surface of an NK cell (e.g., an anti-human CD16a scFv for a BiKE or an anti-human CD16a scFv and an anti-human CD16, NKG2D, NKG2C, NKp46, NKp30, or NKp44 scFv for a TriKE).

In some cases, a cell engager (e.g., a BiTE) targeting a CSPG4 polypeptide can be designed to include an scFv having a heavy chain variable domain comprising SEQ ID NO:17, SEQ ID NO:18, and SEQ ID NO:19, followed by a linker such as (SGGGG)₃₋₅ (SEQ ID NO:35), followed by a light chain variable domain comprising SEQ ID NO:25, SEQ ID NO:26, and SEQ ID NO:27, followed by a linker such as a hinge/linker, followed by an antigen binding domain having the ability to bind to a polypeptide expressed on the surface of a T cell (e.g., an anti-human CD3 scFv).

In some cases, a cell engager (e.g., a BiTE) targeting a CSPG4 polypeptide can be designed to include an scFv having a light chain variable domain comprising SEQ ID NO:25, SEQ ID NO:26, and SEQ ID NO:27, followed by a linker such as (SGGGG)₃₋₅ (SEQ ID NO:35), followed by a heavy chain variable domain comprising SEQ ID NO:17, SEQ ID NO:18, and SEQ ID NO:19, followed by a linker such as a hinge/linker, followed by an antigen binding domain having the ability to bind to a polypeptide expressed on the surface of a T cell (e.g., an anti-human CD3 scFv).

In some cases, a cell engager (e.g., a BiTE) targeting a CSPG4 polypeptide can be designed to include an scFv having a heavy chain variable domain comprising SEQ ID NO:24, followed by a linker such as (SGGGG)₃₋₅ (SEQ ID NO:35), followed by a light chain variable domain comprising SEQ ID NO:32, followed by a linker such as a hinge/linker, followed by an antigen binding domain having the ability to bind to a polypeptide expressed on the surface of a T cell (e.g., an anti-human CD3 scFv).

In some cases, a cell engager (e.g., a BiTE) targeting a CSPG4 polypeptide can be designed to include an scFv having a light chain variable domain comprising SEQ ID NO:32, followed by a linker such as (SGGGG)₃₋₅ (SEQ ID NO:35), followed by a heavy chain variable domain comprising SEQ ID NO:24, followed by a linker such as a hinge/linker, followed by an antigen binding domain having the ability to bind to a polypeptide expressed on the surface of a T cell (e.g., an anti-human CD3 scFv).

In some cases, a cell engager (e.g., a BiKE or a TriKE) targeting a CSPG4 polypeptide can be designed to include an scFv having a heavy chain variable domain comprising SEQ ID NO:17, SEQ ID NO:18, and SEQ ID NO:19, followed by a linker such as (SGGGG)₃₋₅ (SEQ ID NO:35), followed by a light chain variable domain comprising SEQ ID NO:25, SEQ ID NO:26, and SEQ ID NO:27, followed by a linker such as a hinge/linker, followed by one or more antigen binding domains having the ability to bind to a polypeptide expressed on the surface of an NK cell (e.g., an anti-human CD16a scFv for a BiKE or an anti-human CD16a scFv and an anti-human CD16, NKG2D, NKG2C, NKp46, NKp30, or NKp44 scFv for a TriKE).

In some cases, a cell engager (e.g., a BiKE or a TriKE) targeting a CSPG4 polypeptide can be designed to include an scFv having a light chain variable domain comprising SEQ ID NO:25, SEQ ID NO:26, and SEQ ID NO:27, followed by a linker such as (SGGGG)₃₋₅ (SEQ ID NO:35), followed by a heavy chain variable domain comprising SEQ ID NO:17, SEQ ID NO:18, and SEQ ID NO:19, followed by a linker such as a hinge/linker, followed by one or more antigen binding domains having the ability to bind to a polypeptide expressed on the surface of an NK cell (e.g., an anti-human CD16a scFv for a BiKE or an anti-human CD16a scFv and an anti-human CD16, NKG2D, NKG2C, NKp46, NKp30, NKp44 scFv for a TriKE).

In some cases, a cell engager (e.g., a BiKE or a TriKE) targeting a CSPG4 polypeptide can be designed to include an scFv having a heavy chain variable domain comprising SEQ ID NO:24, followed by a linker such as (SGGGG)₃₋₅ (SEQ ID NO:35), followed by a light chain variable domain comprising SEQ ID NO:32, followed by a linker such as a hinge/linker, followed by one or more antigen binding domains having the ability to bind to a polypeptide expressed on the surface of an NK cell (e.g., an anti-human CD16a scFv for a BiKE or an anti-human CD16a scFv and an anti-human CD16, NKG2D, NKG2C, NKp46, NKp30, or NKp44 scFv for a TriKE).

In some cases, a cell engager (e.g., a BiKE or a TriKE) targeting a CSPG4 polypeptide can be designed to include an scFv having a light chain variable domain comprising SEQ ID NO:32, followed by a linker such as (SGGGG)₃₋₅ (SEQ ID NO:35), followed by a heavy chain variable domain comprising SEQ ID NO:24, followed by a linker such as a hinge/linker, followed by one or more antigen binding domains having the ability to bind to a polypeptide expressed on the surface of an NK cell (e.g., an anti-human CD16a scFv for a BiKE or an anti-human CD16a scFv and an anti-human CD16, NKG2D, NKG2C, NKp46, NKp30, or NKp44 scFv for a TriKE).

In some cases, a cell engager (e.g., a BiTE) targeting a CSPG4 polypeptide can be designed to include an IgG (e.g., IgG1) configuration having (a) a heavy chain comprising, consisting essentially of, or consisting of a heavy chain variable domain comprising SEQ ID NO:1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO: 10, and SEQ ID NO:11, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiTE) targeting a CSPG4 polypeptide can be designed to include an IgG (e.g., IgG₁) configuration having (a) a heavy chain comprising, consisting essentially of, or consisting of a heavy chain variable domain comprising SEQ ID NO:8, an Ig hinge, and constant domains (e.g., CH1, CH2, and CH3 domains) and (b) a light chain comprising, consisting essentially of, or consisting of a light chain variable domain comprising SEQ ID NO:16, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiKE) targeting a CSPG4 polypeptide can be designed to include an IgG (e.g., IgG₁) configuration having (a) a heavy chain comprising, consisting essentially of, or consisting of a heavy chain variable domain comprising SEQ ID NO:1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO:10, and SEQ ID NO:11, a constant domain (e.g., a kappa or lambda constant domain), and an antigen binding domain having the ability to bind to a polypeptide expressed on the surface of an NK cell (e.g., an anti-human CD16a scFv).

In some cases, a cell engager (e.g., a BiKE) targeting a CSPG4 polypeptide can be designed to include an IgG (e.g., IgG₁) configuration having (a) a heavy chain comprising, consisting essentially of, or consisting of a heavy chain variable domain comprising SEQ ID NO:8, an Ig hinge, and constant domains (e.g., CH1, CH2, and CH3 domains) and (b) a light chain comprising, consisting essentially of, or consisting of a light chain variable domain comprising SEQ ID NO:16, a constant domain (e.g., a kappa or lambda constant domain), and an antigen binding domain having the ability to bind to a polypeptide expressed on the surface of an NK cell (e.g., an anti-human CD16a scFv).

In some cases, a cell engager (e.g., a BiTE) targeting a CSPG4 polypeptide can be designed to include an IgG (e.g., IgG₁) configuration having (a) a heavy chain comprising, consisting essentially of, or consisting of a heavy chain variable domain comprising SEQ ID NO:17, SEQ ID NO:18, and SEQ ID NO:19, an Ig hinge, and constant domains (e.g., CH1, CH2, and CH3 domains) and (b) a light chain comprising, consisting essentially of, or consisting of a light chain variable domain comprising SEQ ID NO:25, SEQ ID NO:26, and SEQ ID NO:27, a constant domain (e.g., a kappa or lambda constant domain), and an antigen binding domain having the ability to bind to a polypeptide expressed on the surface of a T cell (e.g., an anti-human CD3 scFv). In some cases, a cell engager (e.g., a BiTE) targeting a CSPG4 polypeptide can be designed to include an IgG (e.g., IgG₁) configuration having (a) a heavy chain comprising, consisting essentially of, or consisting of a heavy chain variable domain comprising SEQ ID NO:24, an Ig hinge, and constant domains (e.g., CH1, CH2, and CH3 domains) and (b) a light chain comprising, consisting essentially of, or consisting of a light chain variable domain comprising SEQ ID NO:32, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiKE) targeting a CSPG4 polypeptide can be designed to include an IgG (e.g., IgG₁) configuration having (a) a heavy chain comprising, consisting essentially of, or consisting of a heavy chain variable domain comprising SEQ ID NO:17, SEQ ID NO:18, and SEQ ID NO:19, an Ig hinge, and constant domains (e.g., CH1, CH2, and CH3 domains) and (b) a light chain comprising, consisting essentially of, or consisting of a light chain variable domain comprising SEQ ID NO:25, SEQ ID NO:26, and SEQ ID NO:27, a constant domain (e.g., a kappa or lambda constant domain), and an antigen binding domain having the ability to bind to a polypeptide expressed on the surface of an NK cell (e.g., an anti-human CD16a scFv or an anti-human CD16, NKG2D, NKG2C, NKp46, NKp30, NKp44scFv).

In some cases, a cell engager (e.g., a BiKE) targeting a CSPG4 polypeptide can be designed to include an IgG (e.g., IgG₁) configuration having (a) a heavy chain comprising, consisting essentially of, or consisting of a heavy chain variable domain comprising SEQ ID NO:24, an Ig hinge, and constant domains (e.g., CH1, CH2, and CH3 domains) and (b) a light chain comprising, consisting essentially of, or consisting of a light chain variable domain comprising SEQ ID NO:32, a constant domain (e.g., a kappa or lambda constant domain), and an antigen binding domain having the ability to bind to a polypeptide expressed on the surface of an NK cell (e.g., an anti-human CD16a scFv or an anti-human CD16, NKG2D, NKG2C, NKp46, NKp30, or NKp44 scFv).

In one embodiment, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (i) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications); and/or (ii) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications).

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) and (a) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications) and/or (b) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO:12 (or a variant of SEQ ID NO: 12 with one, two, three, four, five, six, seven, eight, nine, ten, or more amino acid modifications), a framework region 2 having the amino acid sequence set forth in SEQ ID NO:13 (or a variant of SEQ ID NO:13 with one, two, three, four, five, six, seven, eight, nine, ten, or more amino acid modifications), a framework region 3 having the amino acid sequence set forth in SEQ ID NO:14 (or a variant of SEQ ID NO:14 with one, two, three, four, five, six, seven, eight, nine, ten, or more amino acid modifications), and a framework region 4 having the amino acid sequence set forth in SEQ ID NO:15 (or a variant of SEQ ID NO:15 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in Example 8 or Example 9 can be designed to include framework regions or can be designed to include one or more framework regions from another antibody, antibody fragment, or antibody domain.

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (a) a heavy chain variable domain that includes an amino acid sequence having at least 90 percent identity to the amino acid sequence set forth in SEQ ID NO:8 and/or (b) a light chain variable domain that includes an amino acid sequence having at least 90 percent identity to the amino acid sequence set forth in SEQ ID NO:16. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8 and/or (b) a light chain variable domain that includes an amino acid sequence having at least 90, 91, 92, 93, 94, 95, 96, 97, 98, or 99 percent identity to the amino acid sequence set forth in SEQ ID NO:16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO:16.

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (a) a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90 percent identity to the amino acid sequence set forth in SEQ ID NO:16, provided that the light chain variable domain includes the amino acid sequences set forth in SEQ ID NOs:9, 10, and 11. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90, 91, 92, 93, 94, 95, 96, 97, 98, or 99 percent identity to the amino acid sequence set forth in SEQ ID NO:16, provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:16 or the amino acid set forth in SEQ ID NO:16 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CSPG4 polypeptide (e.g., a human CSPG4 polypeptide), can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:1, 2, and 3, and can include a light chain variable domain having the amino acid sequence set forth in SEQ ID NO:16 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (a) a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO: 10, and (iii) a CDR3 that comprises, consists essentially of, or consists of the amino acid sequence set forth in SEQ ID NO:11.

As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO:1, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR3 that consists essentially of the amino acid sequence set forth in SEQ ID NO:3” is a CDR3 that has zero or one amino acid substitutions within SEQ ID NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR1 that consists essentially of the amino acid sequence set forth in SEQ ID NO:9” is a CDR1 that has zero, one, or two amino acid substitutions within SEQ ID NO:9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR2 that consists essentially of the amino acid sequence set forth in SEQ ID NO:10” is a CDR2 that has zero, one, or two amino acid substitutions within SEQ ID NO:10, that has zero, one, two, three, four, or five amino acid residues directly preceding SEQ ID NO:10, and/or that has zero, one, two, three, four, or five amino acid residues directly following SEQ ID NO:10, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR3 that consists essentially of the amino acid sequence set forth in SEQ ID NO:11” is a CDR3 that has zero, one, or two amino acid substitutions within SEQ ID NO:11, that has zero, one, two, three, four, or five amino acid residues directly preceding SEQ ID NO:11, and/or that has zero, one, two, three, four, or five amino acid residues directly following SEQ ID NO:11, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

In another embodiment, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (i) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications); and/or (ii) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications).

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) and having (a) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications) and/or (b) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications) can include any appropriate framework regions.

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (a) a heavy chain variable domain that includes an amino acid sequence having at least 90 percent identity to the amino acid sequence set forth in SEQ ID NO:24 and/or (b) a light chain variable domain that includes an amino acid sequence having at least 90 percent identity to the amino acid sequence set forth in SEQ ID NO:32. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24 and/or (b) a light chain variable domain that includes an amino acid sequence having at least 90, 91, 92, 93, 94, 95, 96, 97, 98, or 99 percent identity to the amino acid sequence set forth in SEQ ID NO:32. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:24 and/or (b) a light chain variable domain that includes an amino acid sequence having 100 percent identity to the amino acid sequence set forth in SEQ ID NO:32.

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (a) a heavy chain variable domain that includes an amino acid sequence having at least 90 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90 percent identity to the amino acid sequence set forth in SEQ ID NO:32, provided that the light chain variable domain includes the amino acid sequences set forth in SEQ ID NOs:25, 26, and 27. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90, 91, 92, 93, 94, 95, 96, 97, 98, or 99 percent identity to the amino acid sequence set forth in SEQ ID NO:32, provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (a) a heavy chain variable domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:32 or the amino acid set forth in SEQ ID NO:32 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CSPG4 polypeptide (e.g., a human CSPG4 polypeptide), can include a heavy chain variable domain having the amino acid sequence set forth in SEQ ID NO:24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:17, 18, and 19, and can include a light chain variable domain having the amino acid sequence set forth in SEQ ID NO:32 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CSPG4 polypeptide (e.g., a human CSPG4 polypeptide) can include (a) a heavy chain variable domain comprising (i) a CDR1 that comprises, consists essentially of, or consists of the amino acid sequence set forth in SEQ ID NO:17, (ii) a CDR2 that comprises, consists essentially of, or consists of the amino acid sequence set forth in SEQ ID NO:18, and (iii) a CDR3 that comprises, consists essentially of, or consists of the amino acid sequence set forth in SEQ ID NO:19, and/or (b) a light chain variable domain comprising (i) a CDR1 that comprises, consists essentially of, or consists of the amino acid sequence set forth in SEQ ID NO:25, (ii) a CDR2 that comprises, consists essentially of, or consists of the amino acid sequence set forth in SEQ ID NO:26, and (iii) a CDR3 that comprises, consists essentially of, or consists of the amino acid sequence set forth in SEQ ID NO:27. As used herein, a “CDR1 that consists essentially of the amino acid sequence set forth in SEQ ID NO:17” is a CDR1 that has zero, one, or two amino acid substitutions within SEQ ID NO:17, that has zero, one, two, three, four, or five amino acid residues directly preceding SEQ ID NO:17, and/or that has zero, one, two, three, four, or five amino acid residues directly following SEQ ID NO:17, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR2 that consists essentially of the amino acid sequence set forth in SEQ ID NO:18” is a CDR2 that has zero, one, or two amino acid substitutions within SEQ ID NO:18, that has zero, one, two, three, four, or five amino acid residues directly preceding SEQ ID NO:18, and/or that has zero, one, two, three, four, or five amino acid residues directly following SEQ ID NO:18, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR3 that consists essentially of the amino acid sequence set forth in SEQ ID NO:19” is a CDR3 that has zero, one, or two amino acid substitutions within SEQ ID NO:19, that has zero, one, two, three, four, or five amino acid residues directly preceding SEQ ID NO:19, and/or that has zero, one, two, three, four, or five amino acid residues directly following SEQ ID NO:19, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR1 that consists essentially of the amino acid sequence set forth in SEQ ID NO:25” is a CDR1 that has zero, one, or two amino acid substitutions within SEQ ID NO:25, that has zero, one, two, three, four, or five amino acid residues directly preceding SEQ ID NO:25, and/or that has zero, one, two, three, four, or five amino acid residues directly following SEQ ID NO:25, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR2 that consists essentially of the amino acid sequence set forth in SEQ ID NO:26” is a CDR2 that has zero, one, or two amino acid substitutions within SEQ ID NO:26, that has zero, one, two, three, four, or five amino acid residues directly preceding SEQ ID NO:26, and/or that has zero, one, two, three, four, or five amino acid residues directly following SEQ ID NO:26, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

As used herein, a “CDR3 that consists essentially of the amino acid sequence set forth in SEQ ID NO:27” is a CDR3 that has zero, one, or two amino acid substitutions within SEQ ID NO:27, that has zero, one, two, three, four, or five amino acid residues directly preceding SEQ ID NO:27, and/or that has zero, one, two, three, four, or five amino acid residues directly following SEQ ID NO:27, provided that the binder (e.g., an antibody, antigen binding fragment, antibody domain, a CAR, a cell engager, and/or an ADC) maintains its basic ability to bind to a CSPG4 polypeptide (e.g., a human CSPG4 polypeptide).

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs:1-3 or SEQ ID NOs:17-19 can be directly connected to a light chain variable domain having the CDRs of SEQ ID NOs:9-11 or SEQ ID NOs:25-27, respectively, via a linker sequence. An example of a linker sequence that can be used to connect a heavy chain variable domain and a light chain variable domain to create a scFv include, without limitation, (SGGGG)₃₋₅ (SEQ ID NO:35).

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3: 348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to a CSPG4 polypeptide (e.g., a human CSPG4 polypeptide) are further described in Table 1.

TABLE 1
Representative number of binders.

| | | SEQ ID NOs of | | | SEQ ID NOs of | |
|---|---|---|---|---|---|---|
| | SEQ ID NOs of | Heavy Chain | | SEQ ID NOs of | Light Chain | |
| | Heavy Chain | Variable | SEQ ID NO of | Light Chain | Variable | SEQ ID NO of |
| | Variable | Domain/Region | Heavy Chain | Variable | Domain/Region | Light Chain |
| | Domain/Region | Framework | Variable | Domain/Region | Framework | Variable |
| Antibody | CDRs | Regions | Domain/Region | CDRs | Regions | Domain/Region |
| 7H5A2 | 1, 2, 3 | 4, 5, 6, 7 | 8 | 9, 10, 11 | 12, 13, 14, 15 | 16 |
| 8G5A6 | 17, 18, 19 | 20, 21, 22, 23 | 24 | 25, 26, 27 | 28, 29, 30, 31 | 32 |

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. Examples 8 and 9 contain the nucleic acid sequences encoding the variable domains of exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae/casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NSO cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of a CSPG4 polypeptide (e.g., a human CSPG4 polypeptide). In some cases, a bispecific binder provided herein can be designed to bind to two different epitopes of the same CSPG4 polypeptide (e.g., a human CSPG4 polypeptide). In some cases, a bispecific binder provided herein can bind to a CSPG4 polypeptide (e.g., a human CSPG4 polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to a CSPG4 polypeptide (e.g., a human CSPG4 polypeptide) and (b) at least one drug covalently linked directly or indirectly to that antigen binding domain. Any appropriate binder (e.g., an antibody, antigen binding fragment, and/or antibody domain) provided herein and having the ability to bind to a CSPG4 polypeptide (e.g., a human CSPG4 polypeptide) can be used as an antigen binding domain to make an ADC described herein. For example, any of the binders set forth in Table 1 can be used to make an ADC having the ability to bind to a CSPG4 polypeptide (e.g., a human CSPG4 polypeptide). Examples of drugs that can be used to make an ADC described herein include, without limitation, Calicheamicin, Monomethyl auristatin E (MMAE), Emtansine (DM1), or Exatecan derivative (Dxd). Any appropriate ADC linker can be used to covalently attach one or more drugs to an antigen binding domain having the ability to bind to a CSPG4 polypeptide (e.g., a human CSPG4 polypeptide) to form an ADC provided herein. For example, cleavable or non-cleavable ADC linkers can be used to covalently attach one or more drugs to an antigen binding domain having the ability to bind to a CSPG4 polypeptide (e.g., a human CSPG4 polypeptide) to form an ADC provided herein. Examples of ADC linkers can be used to covalently attach one or more drugs to an antigen binding domain having the ability to bind to a CSPG4 polypeptide (e.g., a human CSPG4 polypeptide) to form an ADC provided herein include, without limitation, ADC disulfide linkers, ADC hydrazone linkers, ADC peptide linkers, ADC thioether linkers, and ADC PEG-containing linkers.

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a heavy chain variable domain such as a heavy chain variable domain as set forth in Example 8 and 9. In another example, an isolated nucleic acid molecule provided herein can include a nucleic acid sequence encoding a light chain variable domain such as a light chain variable domain as set forth in Example 8 and 9. In some cases, an isolated nucleic acid molecule provided herein can include a nucleic acid sequence encoding both (a) a heavy chain variable domain and (b) a light chain variable domain, with or without, encoding a linker polypeptide (e.g., (SGGGG)₃₋₅ (SEQ ID NO:35)). A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokayotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukaryotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, Chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to a CSPG4 polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to a CSPG4 polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) having cancer to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to a CSPG4 polypeptide (e.g., a human CSPG4 polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to a CSPG4 polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to a CSPG4 polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR* cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. In some cases, a cancer that can be treated as described herein can be a blood cancer. In some cases, a cancer that can be treated as described herein can include one or more solid tumors. In some cases, a cancer that can be treated as described herein can be metastatic cancer. In some cases, a cancer that can be treated as described herein can be a recurrent cancer. In some cases, a cancer that can be treated as described herein can be a chemo-resistant cancer. Examples of cancers that can be treated as described herein include, without limitation, ovarian cancer, glioblastoma, melanoma, squamous cell carcinoma, triple negative breast cancer, mesothelioma, osteosarcoma, AML, chordoma or other tumors in which elevated CSPG4 expression is linked to malignant progression or poor outcome. For example, a mammal having ovarian cancer can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer as compared to a control mammal having comparable cancer and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of a CSPG4 polypeptide (e.g., a human CSPG4 polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of a CSPG4 polypeptide (e.g., a human CSPG4 polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express a CSPG4 polypeptide (e.g., a human CSPG4 polypeptide). In some cases, a binder (e.g., an antibody, antigen binding fragment, and/or antibody domain) provided herein can be immobilized, e.g., on a support, and retention of a CSPG4 polypeptide (e.g., a human CSPG4 polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of a CSPG4 polypeptide (e.g., a human CSPG4 polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In. ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### EXAMPLES

### Example 1—Methods and Materials

Cell lines: Multiple human ovarian cancer cell lines were used in these studies, including the following three CSPG4 positive cell lines: HEY (RRID: CVCL_0297), A2780 (RRID: CVCL_0134), and ES-2 (RRID: CVCL_3509). All cell lines were authenticated via STR profiling by the ATCC Cell Line Authentication Service (Manassas, Virginia). Cell lines were routinely screened for mycoplasma using the Universal Mycoplasma Detection Kit (ATCC cat #30-1012K). ES-2 and A2780 cells were cultured in base DMEM medium (Mediatech cat #10-013-cv), HEY cells in base RPMI 1640 medium (Gibco cat #11875-093), supplemented with 10% fetal bovine serum (Atlanta Biologicals cat #SS11150H, Lot #H1810S), 1% penicillin and streptomycin (Gibco cat #15140-122) at 37° C./5% CO₂. Cell lines were routinely used between passages 2-15 from thaw. CSPG4-CRISPR knockout and mock stable transfected variants of each cell line were maintained in the appropriate medium supplemented with 0.6 g/mL puromycin (Sigma cat #P8833).

Generation of CSPG4 CRISPR cell lines: The guide RNA (gRNA) target sequences used to make the CSPG4-CRISPR cells were 5′ CGAGCGCGGCTCTGCTCCTG 3′ (SEQ ID NO:37) and 5′AGAGACCTGGAGACACCAGG 3′ (SEQ ID NO:38). The gRNAs were inserted into plasmid pU6-gRNA. Both gRNA plasmids were co-transfected with plasmid expressing the CAS9 enzyme (pT3.5 Caggs-FLAG-hCas9) as well as plasmids for puromycin and GFP selection, pcDNA-PB7 and pPB SB-CG-LUC-GFP (Puro)(+CRE). Transfection was performed using the Lipofectamine 2000 reagent (Invitrogen cat #11668-019) following the manufacturer's suggested protocol. Mock cell lines were transfected with selection plasmids only (pcDNA-PB7 and pPB SB-CG-LUC-GFP (Puro)(+CRE)) and selected as a pool by culture in puromycin containing medium (0.6 μg/mL). CSPG4-CRISPR knockout cell lines were selected by clonal plating (based on GFP expression in the transfected population) in 96 well plates and culture in puromycin containing media (0.6 μg/mL). Single cell derived colonies were expanded and screened by genomic PCR for the deletion of the CSPG4 gene using primers 5′ GGGCCCTTTAAGAAGGTTGA 3′ (SEQ ID NO:39) and 5′ GTTTTGACAGCCCAAACCAG 3′ (SEQ ID NO:40). Cell lines were further screened by immunoblot and flow cytometry to verify the loss of CSPG4 protein.

Additional antibodies and reagents are listed in Table 2.

TABLE 2
Antibodies and Regents

| Antibody/reagent | Source | Catalog # | RRID: |
|---|---|---|---|
| anti-CSPG4 9.2.27 - | Santa Cruz | sc-80003 | AB_1121950 |
| immunohistochemistry | Biotechnology | | |
| anti-CSPG4 9.2.27 - western | EMD Millipore | MAB2029 | AB_94509 |
| blot and flow cytometry | | | |
| anti-a Tubulin | | cp06 | AB_2617116 |
| Anti-CSPG4 763.74 | Dr. Soldano | | |
| | Ferrone, Dana- | | |
| | Farber Cancer | | |
| | Institute | | |
| anti-TCF8/Zeb1 | Cell Signaling | 3396 | AB_1904164 |
| anti-Vimentin | Technology, Inc. | 5741 | AB_10695459 |
| anti-Claudin-1 | | 13255 | AB_2798163 |
| anti-Slug | | 9585 | AB_2239535 |
| Anti-E-Cadherin | | 3195 | AB_2291471 |
| anti-GAPDH | | 2118 | AB_561053 |
| anti-phospho-FAK(Y397) | | 8556 | AB_10891442 |
| anti-total FAK | | 3285 | AB_2269034 |
| normal mouse IgG1 | MP Biomedical | 50327 | AB_2334246 |
| normal mouse IgG2a | | 50328 | AB_2334247 |
| HRP-donkey-anti-mouse | Jackson | 715-035-150 | AB_2340770 |
| secondary | Immunoreserarch | | |
| HRP-donkey-anti-rabbit | | 711-035-152 | AB_10015282 |
| secondary | | | |
| Cy3-donkey-anti mouse | | 715-166-150 | AB_2340798 |
| secondary | | | |
| FAK inhibitor VS-4718 (PND- | ChemieTek | CT- | |
| 1186) | | VS4718 | |
| Cisplatin | Sigma Aldrich | PHR1624 | |

Transfection of siRNA: Small interfering RNA (siRNA) specific for human ZEB1 (cat #sc-38643) was purchased from Santa Cruz Biotechnology (Dallas, TX), and negative control siRNA was purchased from Qiagen, Inc. (cat #1027281, Germantown, MD). Cells were seeded in six-well plates until they were₆₀-7₀% confluent prior to transfection. Cells were transfected using the Lipofectamine RNAimax transfection reagent (cat #13778075, ThermoFisher Scientific) following the manufacturer's suggested protocol. Cells were harvested 48 hours post-transfection for plating in growth, invasion and spheroid formation assays.

Anchorage independent growth assay: Soft agar growth assays were performed as previously described by Yang, et al., Cancer Res., 69)19):7538-47 (2009) with the following modifications. Cells were plated in the upper agarose layer at a final concentration of 0.6% agarose. Colonies were counted in five random fields/well from triplicate wells at the indicated time point (see figure legends) for each cell line. Experiments were performed a minimum of 3 times and the data shown are the average number of colonies from five fields/well from triplicate wells from three combined experiments, +/−s.e.m. Statistical significance was determined using Students t-test.

Xenograft Intraperitoneal Injection Mouse Model: All animal studies were approved by the University of Minnesota Institutional Animal Care and Use Committee (IACUC 1908-37330A). NOD/SCID/γc−/− (NSG) 12-week-old female mice (Jackson Laboratories, Bar Harbor, ME) were used. One day prior to tumor injection, the mice were sub-lethally irradiated (225 cGy). The following day, 4 female mice were injected intraperitoneally with 2×10⁵ A2780 Mock luciferase expressing tumor cells and 5 female mice were injected intraperitoneally with 2×10⁵ A2780 CSPG4-CRISPR luciferase expressing tumor cells. Tumor burden was monitored post luciferin injection (Goldbio, St. Louis, MO) using bioluminescent imaging (BLI) using the IVIS Spectrum in vivo Imaging system (PerkinElmer) on Day 6, 13, and 27 post cell injection. Image analysis was performed using Living Image 4.5 software (PerkinElmer).

Cell Invasion Assays: Cells (2.5-5.0×10⁴) in normal growth medium were added to the top chamber of triplicate wells of matrigel invasion chambers (8 μm, Corning, NY) and the bottom chambers filled with complete medium (ES-2, A2780) or serum free medium (HEY) and cultured for 16-24 hours in a tissue culture incubator at 37° C., 5% C02 atmosphere. Remaining cells in the upper chamber were removed with a cotton swab and the invaded cells fixed and stained using Differential Quick Staining Kit (Electron Microscopy Sciences, Hatfield, PA). The invaded cells were enumerated under a microscope with 100× magnification from five random fields/well. Each experiment was repeated a minimum of three times.

SpheroidFormation Assays: 2×10⁵ cells were suspended in 1% high viscosity methylcellulose (Sigma cat #M0512) diluted in complete growth media, plated in 6-well plates coated with Poly-HEMA and cultured for 7 days. At the indicated times, spheroids were imaged and the spheroids with a diameter over 100 μm are enumerated under a microscope with 100× magnification from five random fields/well. Spheroids grown in methylcellulose culture were harvested by dilution-dispersion in PBS, centrifugation at 400×g for 15 minutes, and washed twice in PBS for subsequent analysis. Each experiment was repeated a minimum of three times.

Cisplatin cytotoxicityAssay: Cisplatin stock (3.3 mM) was diluted with growth medium to the required concentrations before each experiment. Cells were seeded into 96-well plates at 1.0×10³ cells/well. The following day media was removed from wells and replaced with 100 μl media containing the indicated treatment or media alone (baseline) in triplicate wells. After 96 hours of treatment, 201 of MTS reagent (Promega, cat #G3580) was added to each well and plates were incubated in the dark for 2 hours at 37° C., 5% C02 atmosphere. Absorbance at 570 nm was collected on a Tecan 200 plate reader. Each experiment was repeated a minimum of three times.

RNA seq analysis: Total RNA was isolated from two technical replicates from the ES-2 parent, Mock, and CSPG4-CRISPR cell lines using the Rneasy RNA isolation kit (Qiagen, cat #74104) following the manufacturer's suggested protocol. RNA samples were submitted to the University of Minnesota Genomics Center for quality control assessment on an Agilent Bioanalyzer and quantification using a fluorimetric RiboGreen assay. A strand-specific RNA-seq library was generated and sequenced on an IlluminaiSeq 2500 in high output mode, ˜20million reads/sample (duplicate samples) with 2×125 bp paired end reads. Bulk RNAseq samples were processed and aligned using the CHURP version 0.2.2 command line interface framework. A full description of the CHURP pipeline can be found in Baller, et al., CHURP: A lightweight CLI Framework to Enable Novice Users to Analyze Sequencing Datasets in Parallel. Briefly, trimmomatic version 0.33 (see Bolger, et al., Bioinformatics, 30(15):2114-20 (2014)) was used to clean reads for adapter contamination and low-quality sequence. FastQC (see Andrews, FastQC: A Quality Control Tool for High Throughput Sequence) was used to generate sequence quality reports for raw and trimmed reads. HISAT2 version 2.1.0 (see Kim, et al., Nat Biotechnol., 37(8):907-15 (2019)) was used to align samples to the genome reference consortium H. sapiens build 38 reference genome. FeatureCounts v1.6.2 was used to count mapped reads to genes. See, Liao, et al., Bioinformatics, 30(7):923-30 (2014).

Gene expression and pathway analysis: All differential gene expression and pathway analyses were done in R v 3.6.3 (R Core Team, 2020). Differential gene expression analysis was done using EdgeR v 3.28.1. See Robinson, et al., Bioinformatics, 26(1):139-140 (2010). Differentially expressed genes were identified between the ES2 CSPG4-CRISPR/Cas9 knock-out cell line and the average of the ES-2 parent and mock cell lines. Counts were normalized using the relative log expression normalization method and only genes with counts per million greater than one in two or more samples were kept. A general linear model approach was used to test for differentially expressed genes. A gene was categorized as differentially expressed if the p-value was less than 0.01 after p-value adjustment and log 2 fold change was greater than one. P-values were adjusted using the Benjamini & Hochberg method. GO term enrichment analysis and gene set enrichment analysis (GSEA) were done using the ClusterProfler R package. See, Yu, et al., OMICS, 16(5):284-7 (2012). The hallmark gene set from the Molecular Signatures Database v 7.1 (gsea-msigdb.org/gsea/msigdb/index.jsp) was used in the GSEA.

Survival analysis was performed using the Kaplan-Meier Plotter web-based informatics tool. See, Gyorffy, et al., Endocr Relat Cancer. 19(2):197-208 (2012); and Nagy, et al., Sci Rep. 8(1):9227 (2018). A total of fifteen ovarian cancer patient cohorts were included in the combined analysis, including the ovarian cancer TCGA cohort and 14 additional cohorts from the Gene Expression Omnibus (GEO) database; most cases in these datasets are serous histology, with a small proportion of endometrioid type ovarian cancers. JetSet optimal probes (see, Gyorffy, et al., Breast Cancer Res Treat. 123(3):725-31 (2010)) were selected for CSPG4 (MCSPG) and ZEB1 expression analysis; 1656 patients had available data for CSPG4, while 355 patients had available data for ZEBl. The cohort was separated by the median for either normalized single gene expression or the mean of normalized combined expression of both genes. Outcomes were censured at 5 years to harmonize with the duration of follow up for separately performed immunohistochemical studies.

Analysis of EMT signature enrichment was performed using the web based Xena informatics tool (see Goldman, et al., Nat Biotechnol. 38(6):675-8 (2020)) on the ovarian cancer TCGA dataset. The cohort was separated by mean CSPG4 gene expression, and EMT signature score was calculated using Xena genomic signatures feature. See, Salt et al., Cancer Discov. 4(2):186-99 (2014).

Western blot: Western blots were performed using standard methods as described previously by Yang, et al., Cancer Res. 69(19):7538-47 (2009).

Confocal microscopy: Cells were plated on coverslips for 48 hours, fixed with 4% paraformaldehyde for 15 minutes, permeabilized with 0.05% Triton X-100 for 5 min at room temperature, and blocked with 1% donkey serum for 1 hour. Coverslips were incubated with the indicated primary antibodies at 1 g/mL overnight at 4° C., washed twice with PBS+1% BSA for 10 minutes at room temperature, and incubated with Cy3-conjugated anti-mouse secondary antibody (1:5000) for 1 hour at room temperature. After washing cells twice with PBS+1% BSA, images were captured as described by Yang, et al., J Cell Biol. 165(6):881-91 (2004).

Flow cytometry: Cells were released in PBS/5 mM EDTA solution and washed 2 times with FACS buffer (RPMI media supplemented with 1% goat serum and 5 mM HEPES). Cells were incubated with the indicated primary antibody for 45 minutes at 4° C., washed 3 times with FACS buffer, and then incubated with goat anti-mouse phycoerythrin-conjugated secondary antibody for 30 minutes at 4° C. Antibody staining was analyzed on a BD Biosciences Accuri C6 flow cytometry system and data graphed using the Accuri C6 software (BD Biosciences).

Generation of novel CSPG4 antibody: CSPG4-specific mouse monoclonal antibody 7H5A2 was generated by Promab Biotechnologies Inc (Richmond, CA) by injection of a recombinant CSPG4 protein immunogen corresponding to aa 1538-2221 of the CSPG4 core protein extracellular domain, expressed and purified from a eukaryotic expression system (see FIG. 1A). The specificity of the antibody was determined by screening against CSPG4 wild type and knockout cell lysates via western blot and cell staining via immunofluorescence (see FIGS. 1B and 1C). 7H5A2 is isotype IgG1.

Ovarian cancer patient tissue cohort: The cohort consists of 126 epithelial ovarian cancer patients with long-term clinical follow-up, who have undergone initial surgery and treatment at the Hunan Cancer Hospital, affiliated to Xiangya School of Medicine of Central South University of China, a specialized cancer hospital certified by the Joint Commission International (JCI). Inclusion criteria for the ovarian cancer patient cohort were histologically confirmed epithelial ovarian carcinoma including three major histopathologic subtypes (serous, mucinous, and other adenocarcinoma); treatment with platinum/taxane based chemotherapy after debulking surgery; no radiotherapy or biological therapy before surgery; and Karnofsky Performance Status score ≥80 prior to surgery. Patients were staged according to the International Federation of Gynecology and Obstetrics (FIGO) surgical staging system. Another 16 patients with benign ovarian lesions and 26 hysterectomy patients with normal ovarian tissues were also recruited.

Protocols are approved by the Ethics Committee of Hunan Cancer Hospital (Changsha, China) and all patients provided written informed consent on file with the hospital.

Immunohistochemistry: The specimens were paraffin embedded and the tissue sections (4 m) dewaxed, rehydrated, blocked with 3% BSA, and subjected to antigen retrieval. After washing, the sections were incubated with antibody 9.2.27 against CSPG4 (1:1000) at 4° C. overnight. Mouse IgG (cat #A7028, Beyotime, Shanghai, China) was used as a negative control. The bound antibodies were detected using horseradish peroxidase (HRP)-conjugated goat anti-mouse IgG (Beyotime cat #A0216, China) and visualized by DAB (DAB-2031, Maixin Biotech. Co., Fuzhou, China), followed by counterstaining with hematoxylin (CTS-1090, Maixin Biotech. Co., Fuzhou, China). The results were evaluated under a microscope by two pathologists in a blinded manner.

Statistical analysis: All statistical analysis was performed with Graphpad PRISM 6 (Graphpad Software, San Diego, CA) unless otherwise indicated. Data are graphed as the mean s.e.m. unless otherwise indicated. Comparison of two independent samples was done utilizing two-tailed Student's t-test with Welch's correction. Differences in tumor burden over time within the xenograft model were analyzed by ordinary two-way ANOVA with Sidak's multiple comparison test. Values of p<0.05 were considered statistically significant. Overall survival and disease-free survival curves for patients with low and high CSPG4 expression in tumor specimens were analyzed using the Kaplan-Meier method and compared using the log-rank test (SPSS 15.0 software, Chicago, IL, USA). Univariate and multivariate analysis were performed using Cox regression model after adjusting for baseline characteristics. p<0.05 was considered statistically significant.

Example 2—CSPG4 is a Protein Biomarker of Poor Survival in Ovarian Cancer Patients.

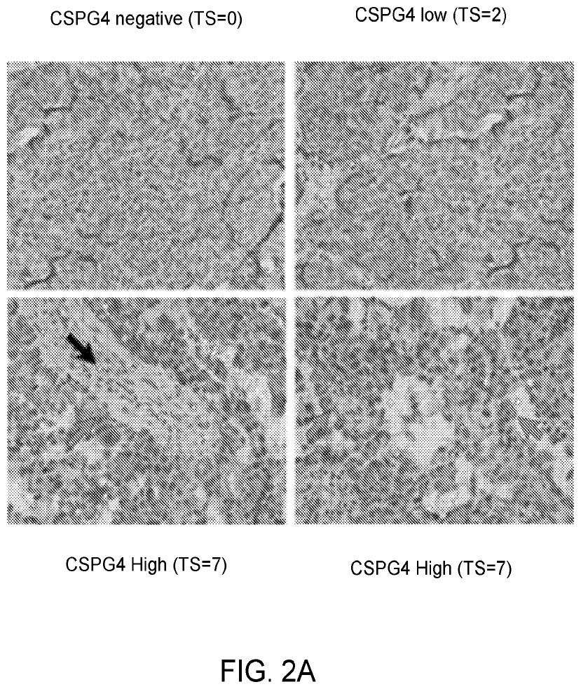

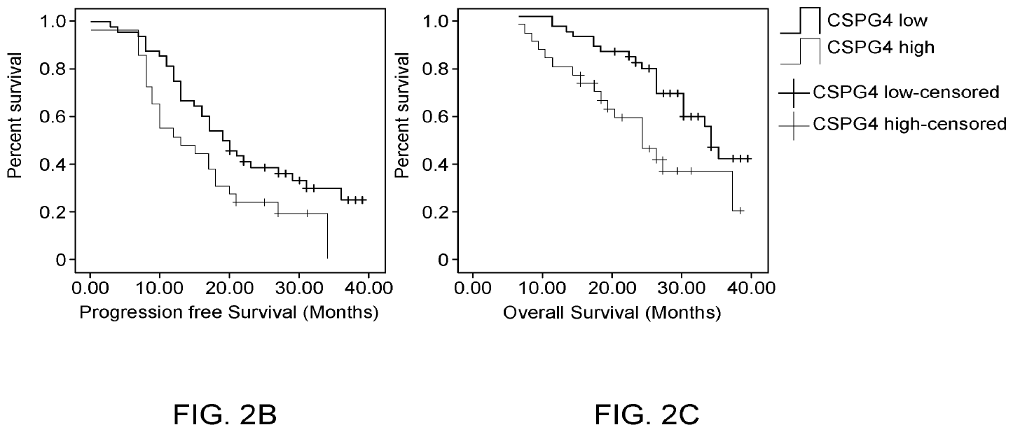

CSPG4 protein expression was evaluated by immunohistochemistry (TIC) in a cohort of 126 ovarian cancer patients (FIG. 2). CSPG4 staining intensity and fraction of tumor cells positive were scored by pathologists blinded to patient ID. CSPG4 protein expression is significantly higher in ovarian cancer (39.68%, 50/126) compared to benign (12.5%, 2/16) or normal ovarian tissue (11.54% 3/26) (X²=11.04, P=0.004) (Table 3).

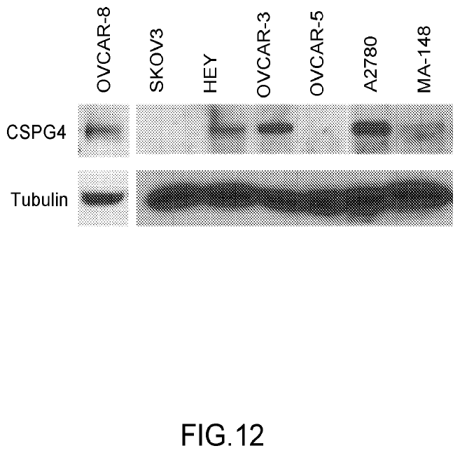

TABLE 3
CSPG4 expression in ovarian cancer, benign ovarian lesions
and normal ovarian tissues. CSPG4 expression is enriched
in malignant tumor tissue vs. normal and benign ovarian tissue.
CSPG4 high vs. low expression is defined in FIG. 12

| | | | | High | | |
|---|---|---|---|---|---|---|
| | | CSPG4 | | expression | | |
| Group | N | Low | High | rate (%) | X² | P |
| Malignant | 126 | 76 | 50 | 39.68 | 11.042 | 0.004 |
| Normal | 26 | 23 | 3 | 11.54 | | |
| Benign | 16 | 14 | 2 | 12.5 | | |

CSPG4 was detected in uniform patterns in tumor cells in contact with tumor-associated stroma, however, CSPG4 positivity was more heterogenous in areas distant from stroma (FIG. 2A). Patients with low expression of CSPG4 protein have significantly longer progression free survival (Kaplan-Meier PFS: 22.615±1.754 vs 16.559±1.940, X²=4.316, P=0.038) and improved overall survival (Kaplan-Meier OS: 31.027±1.353 vs 24.046±2.177, X²=7.366, P=0.007) compared to patients with high expression of CSPG4 (FIG. 2B, 2C). The poor prognosis associated with elevated CSPG4 was independent of patient age, tumor subtype (1 or 2), clinical stage (I/II vs. III/IV), degree of differentiation, the presence of omental or lymphatic metastasis or the volume of ascites (see Table 4). Hazard ratio analysis demonstrated that high CSPG4 levels are an independent indicator of poor overall survival in both univariate (HR=2.33; 95% CI 1.230 to 4.427; p=0.009) and multivariate (HR=2.54; 95% CI 1.255-5.140; p=0.010) analyses (see Table 5).

TABLE 4
CSPG4 expression level is an independent prognostic factor
in ovarian cancer. The poor prognosis associated with
elevated CSPG4 is independent of patient age, tumor
subtype (I or II), clinical stage (I/II vs. III/IV),
degree of differentiation, the presence of omental or
lymphatic metastasis or the volume of ascites.

CSPG4

| Variable | N | Low | High | P |
|---|---|---|---|---|
| Age | | | | 0.394 |
| <50 years | 46 | 30 | 16 | |
| >=50 years | 80 | 46 | 34 | |
| Type | | | | 0.995 |
| I | 41 | 25 | 16 | |
| II | 75 | 45 | 30 | |
| None Type I or II | 10 | 6 | 4 | |
| Clinical Stage | | | | 0.190 |
| I + II | 22 | 16 | 6 | |
| III + IV | 104 | 60 | 44 | |
| Differentiation | | | | 0.143 |
| Degree | | | | |
| Low | 109 | 63 | 46 | |
| Middle and High | 17 | 13 | 4 | |
| Omental | | | | 0.116 |
| Metastasis | | | | |
| No | 51 | 35 | 16 | |
| Yes | 75 | 41 | 34 | |
| Lymph Metastasis | | | | 0.524 |
| No | 42 | 27 | 15 | |
| Yes | 62 | 36 | 26 | |
| Not Cleaned | 22 | 13 | 9 | |
| Ascites Volume | | | | 0.562 |
| <500 mL | 59 | 34 | 25 | |

TABLE 5
CSPG4 expression level (High vs. Low) is a significant indicator of
overall patient survival (OS) in both univariate and multivariate analysis.
CSPG4 expression level (high vs. low) was determined by IHC.

| | Univariate Analysis | | | Multivariate Analysis | | |
|---|---|---|---|---|---|---|
| Variables | HR | 95% CI | P | HR | 95% CI | P |
| Age | 1.726 | 0.839-3.549 | 0.138 | 1.244 | 0.586-2.638 | 0.570 |
| Clinical Stage | 4.216 | 1.016-17.506 | 0.048 | 3.634 | 0.657-20.203 | 0.139 |
| Differentiation Degree | 0.578 | 0.225-1.483 | 0.254 | 0.727 | 0.269-1.965 | 0.529 |
| Omental Metastasis | 2.298 | 1.345-6.376 | 0.007 | 1.870 | 0.761-4.597 | 0.172 |
| Lymph Metastasis | 0.972 | 0.612-1.544 | 0.905 | 0.603 | 0.336-1.083 | 0.090 |
| Ascites Volume | 1.499 | 0.776-2.895 | 0.228 | 1.681 | 0.845-3.343 | 0.139 |
| CSPG4 (High/Low) | 2.334 | 1.230-4.427 | 0.009 | 2.540 | 1.255-5.140 | 0.010 |

### Example 3—CSPG4 Promotes EOC Tumor Expansion, Invasion, Cisplatin Resistance

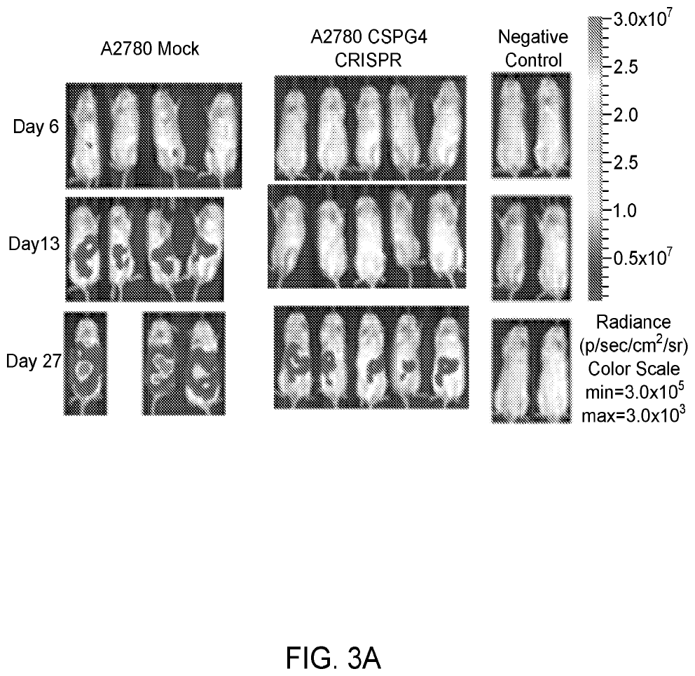

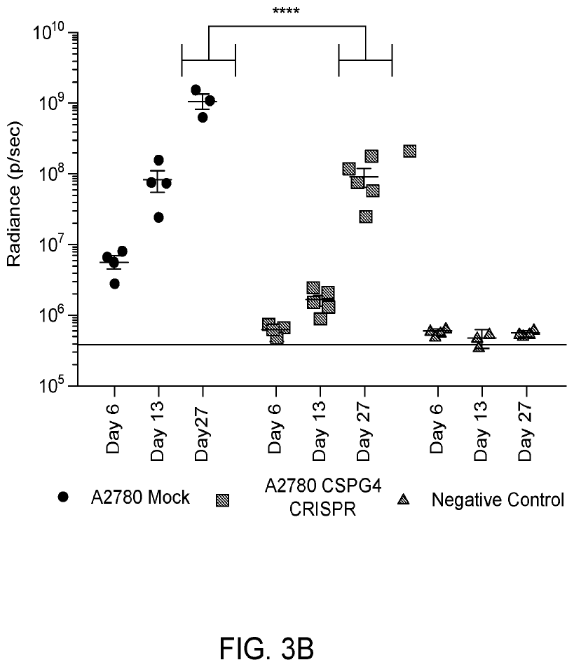

To determine if CSPG4 expression promotes tumor growth in vivo, an IP xenograft injection model was used. Mice were injected with 2×10⁵ mock transfected or CSPG4 knockout A2780 cells. Tumor growth, as monitored by bioluminescence, was significantly reduced in mice receiving CSPG4 knockout cells (FIG. 3A, 3B). By 27 days post injection, CSPG4 expression promoted almost an order of magnitude growth advantage compared to CSPG4 knockout counterparts (FIG. 3A, 3B).

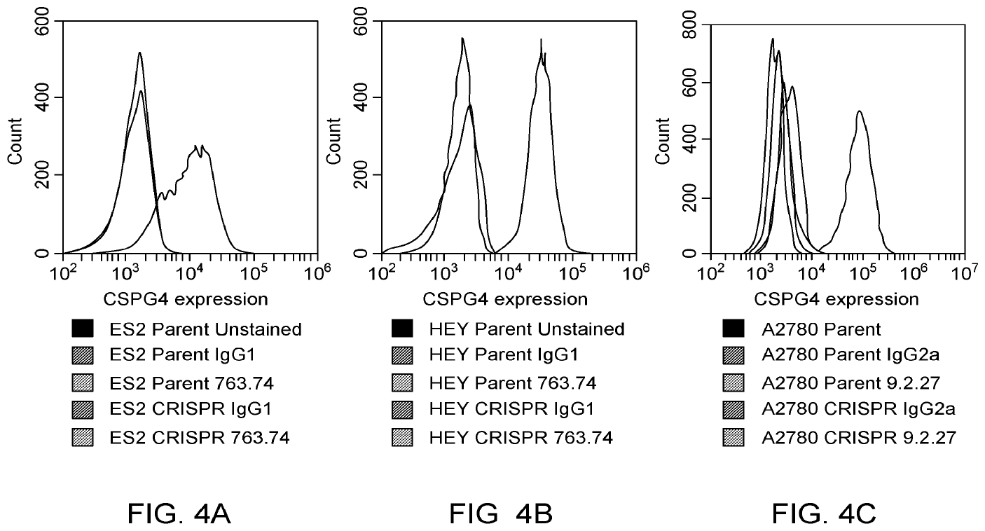

The functional significance of CSPG4 expression in EOC cells was explored using multiple in vitro correlate analyses of malignant phenotypes. Three ovarian cell lines were selected that had high levels of CSPG4 expression out of 11 cell lines screened by confocal analysis, flow cytometry and western blot). See, FIG. 4A-4C. The selected ovarian cancer cell lines (ES-2, HEY, A2780) originated from patients harboring different subtypes of EOC. See, Domcke, et al., Nat Commun. 4, 2126 (2013). The entire CSPG4 locus was deleted in each of these cell lines using CRISPR/Cas9 and verified knockout efficiency using PCR, western blot, and flow cytometry (see FIG. 4A-C).

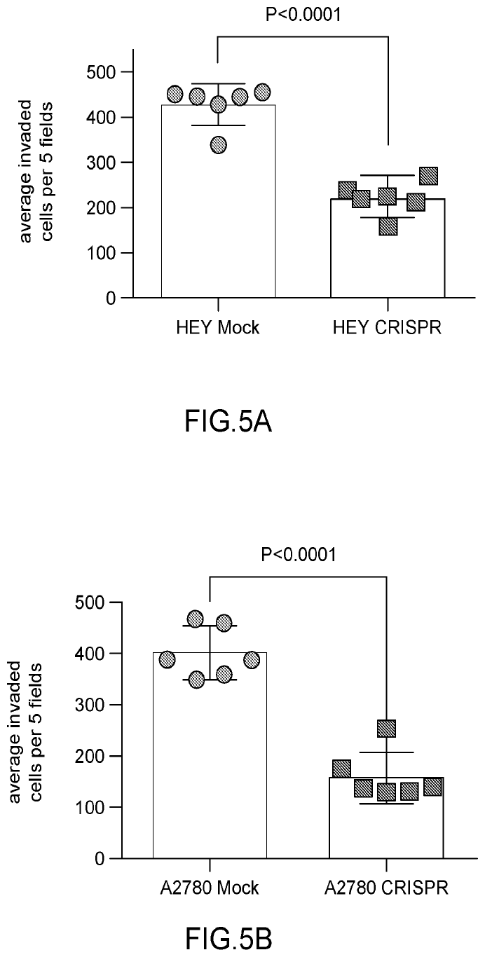

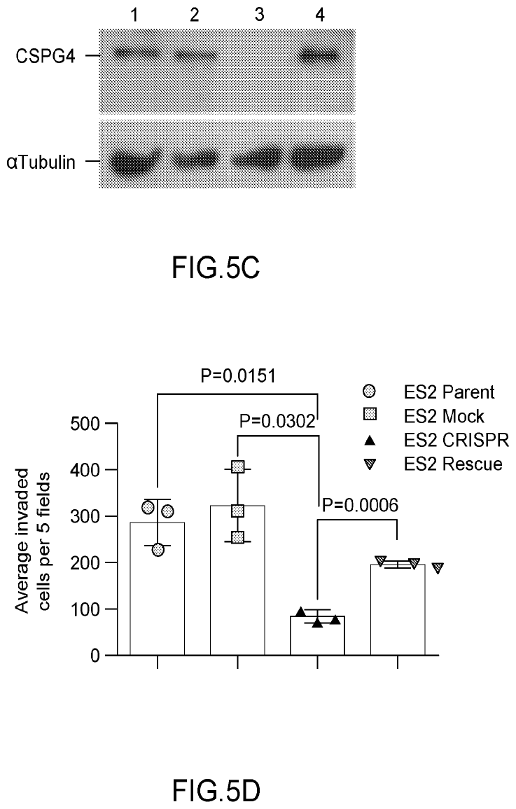

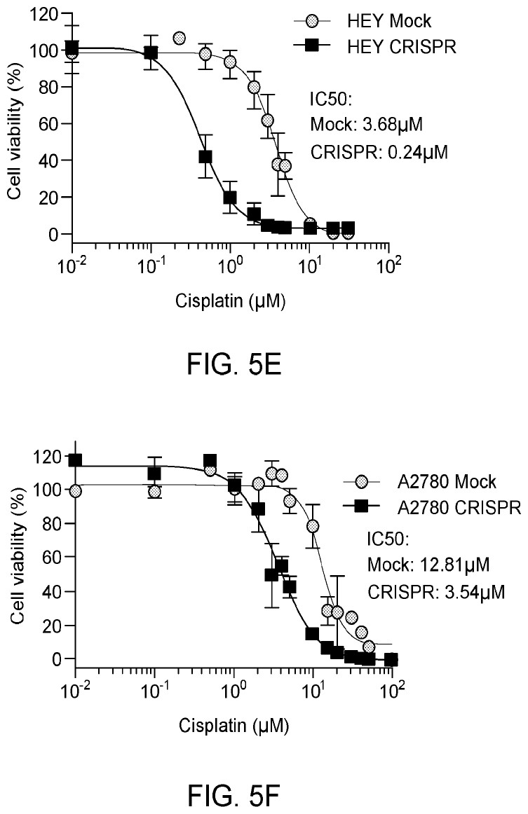

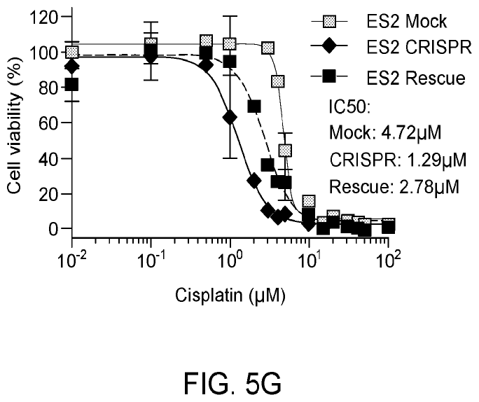

Using a standard matrigel invasion assay, it was determined that loss of CSPG4 expression significantly decreased the invasive phenotype of all three cell lines (FIG. 5A-D). Importantly, the invasive phenotype could be rescued by re-expression of CSPG4 in the ES-2 CRISPR (CSPG4-deleted) cells (FIG. 5C, D).

While platinum-based therapy is widely used as a first-line adjuvant therapy for patients with EOC, recurrence with resistant disease is a common complication that reduces overall survival. To investigate whether CSPG4 expression impacts cisplatin resistance in EOC, the cisplatin sensitivity was evaluated in the cell lines using an MTS assay. Loss of CSPG4 resulted in increased cisplatin sensitivity in all three cell lines, as evidenced by a 3.6-to-8.8-fold reduction in the IC₅₀ for the knockout cell lines (FIG. 5 E-G). As was observed for the invasion phenotype, re-expression of CSPG4 in edited cells resulted in reversal of cisplatin sensitivity (FIG. 5G, purple curve).

### Example 4—CSPG4 Expression Enhances Cell Adhesion, Promotes Spheroid Formation and FAK Activation

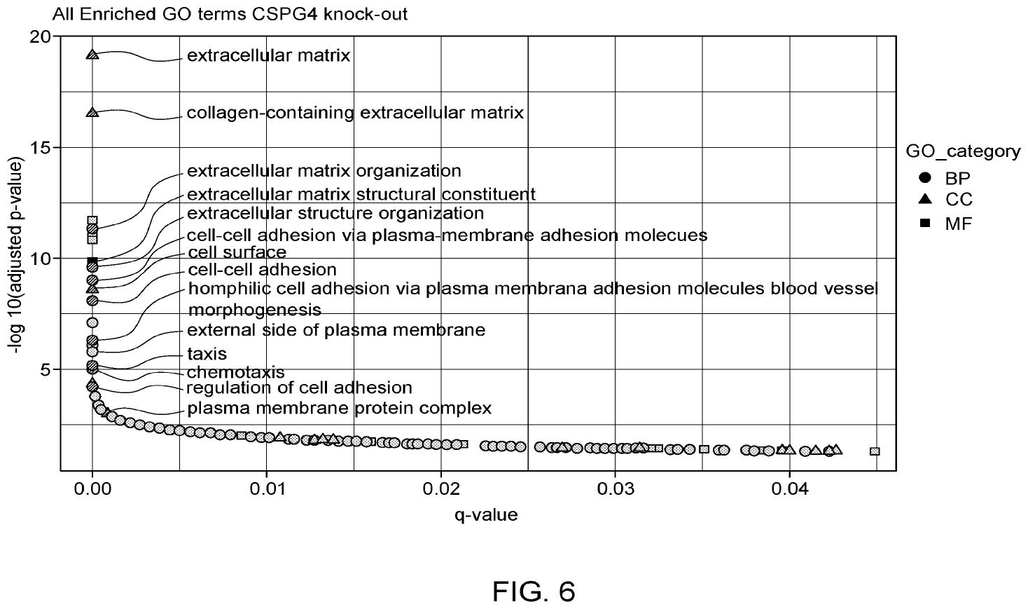

Gene ontology (GO) term enrichment analysis of the set of differentially expressed genes in ES-2 controls and CSPG4 knockout cell lines identifies significant enrichment (adjusted p-value <0.005) for GO terms associated with regulation of cell adhesion, associated signaling pathways and extracellular matrix collagen/organization (FIG. 6). This association supports previous studies which functionally link CSPG4 to activation of integrin and downstream integrin stimulated pathways such as focal adhesion kinase activation. Increased tumor cell adhesion can impact malignant progression in several distinct but overlapping ways. It can promote increased anchorage independence and/or enhance the formation of cellular aggregates (spheroids) within the abdominal cavity. These spheroids, which originate from individual cells that have acquired an anchorage independent phenotype, adhere to mesothelial-lined surfaces and form metastases by invading into sub-mesothelial tissues.

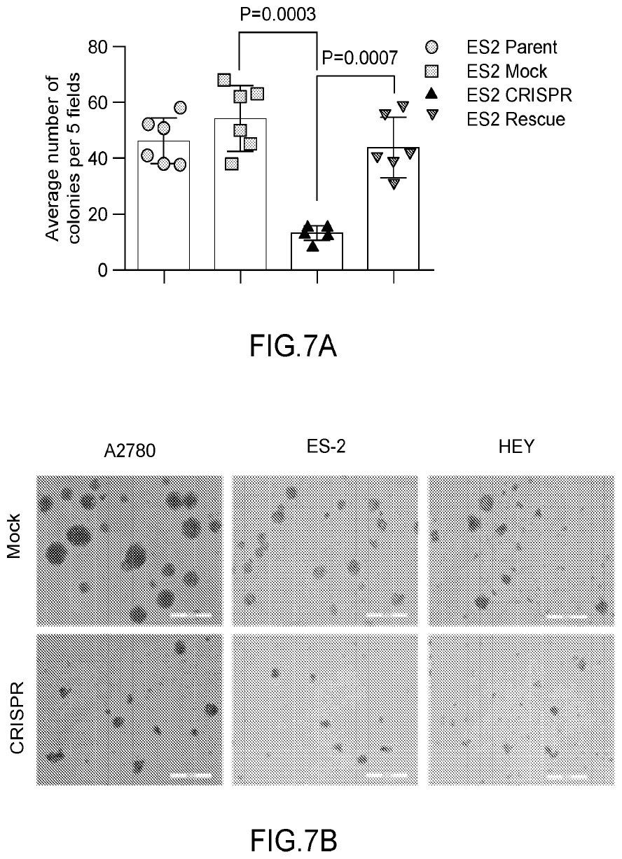

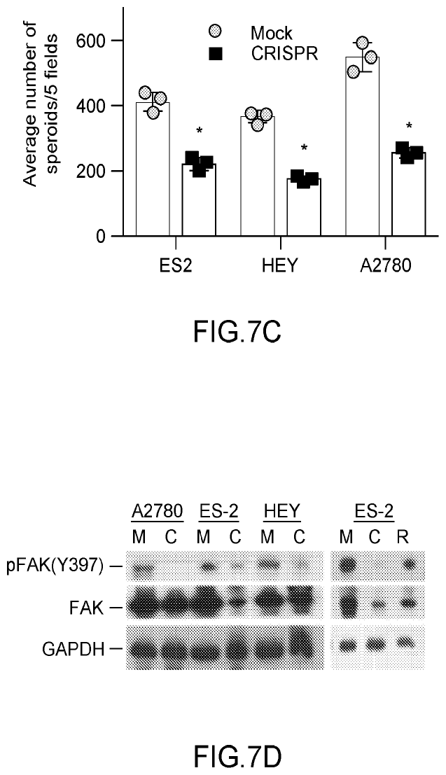

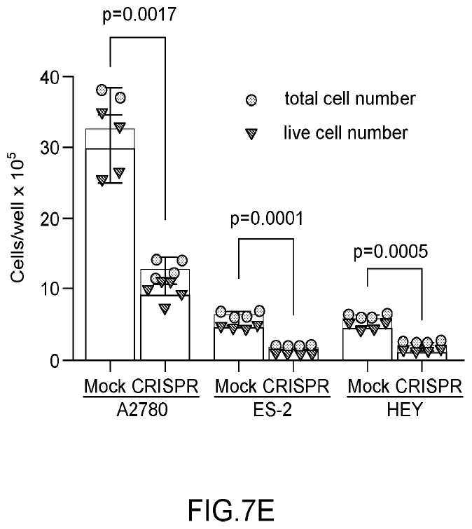

As expected, loss of CSPG4 expression in EOC cells inhibited anchorage independent growth in agarose and spheroid formation by cells cultured in methylcellulose (FIGS. 7A, B and C). Consistent with the GO term enrichment analysis, CSPG4 expression promoted activation of FAK, which was completely inhibited in CSPG4 gene deleted counterparts (FIG. 7D). CSPG4 rescue of the gene edited cells resulted in restoration of FAK activation in gene edited cells (FIG. 7E). While CSPG4 loss inhibited spheroid formation, there was no significant impact on cell survival in the CSPG4 null spheroids (FIG. 7F), indicating the primary impact of inhibiting CSPG4 expression was on cell proliferation within the spheroids. These data suggest that CSPG4 mediated activation of cell adhesion dependent pathways can enhance metastasis by stimulating invasion and spheroid formation in EOC.

### Example 5—CSPG4 Expression is Associated with Mesenchymal Transition in Ovarian Cancer Cells

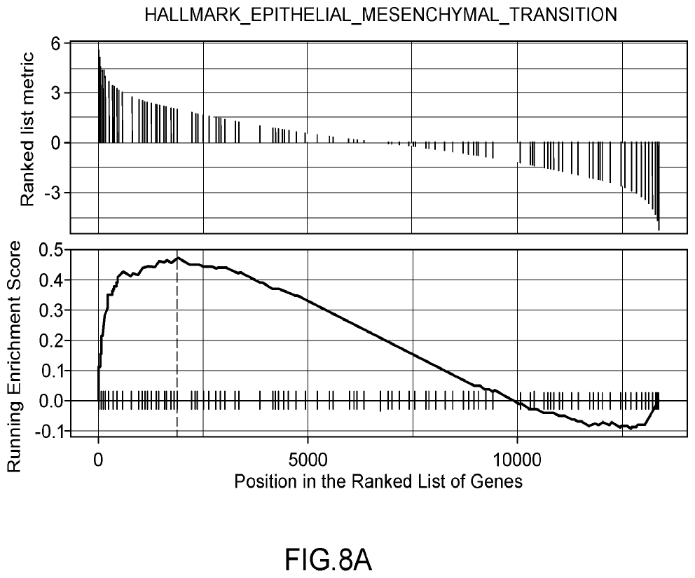

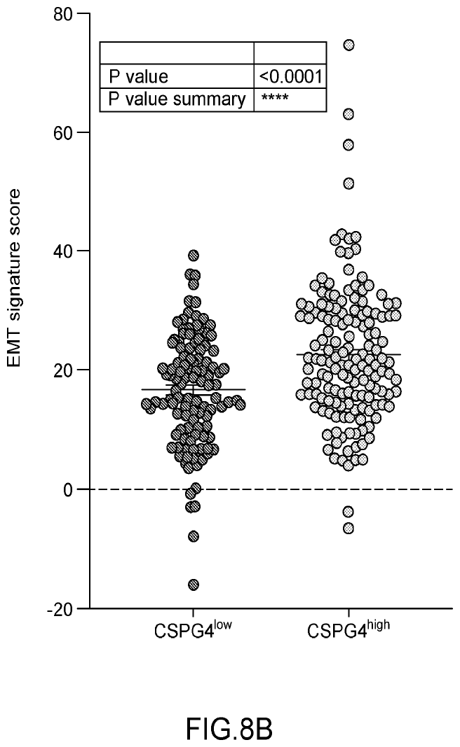

Gene set enrichment analysis (GSEA) of these RNA seq data indicated that CSPG4 deletion impacts the differential expression of several genes within the hallmark epithelial to mesenchymal transition gene set from the Molecular Signatures Database v 7.1 (gsea-msigdb.org/gsea/msigdb/index.jsp) (FIG. 8A). Analysis of a large array of gene expression data from the ovarian cancer TCGA also demonstrates high CSPG4 expression is associated with an EMT signature, leading us to conclude that high CSPG4 portends a more mesenchymal phenotype in EOC tumors (FIG. 8B).

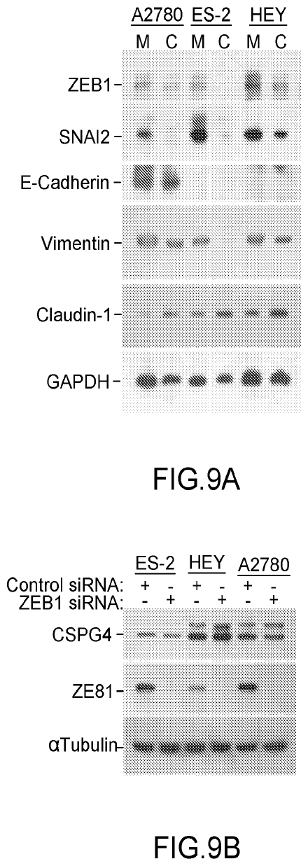

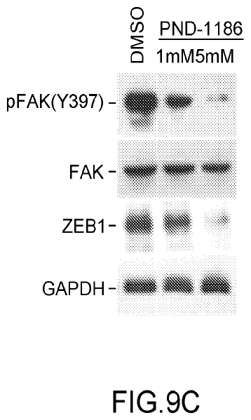

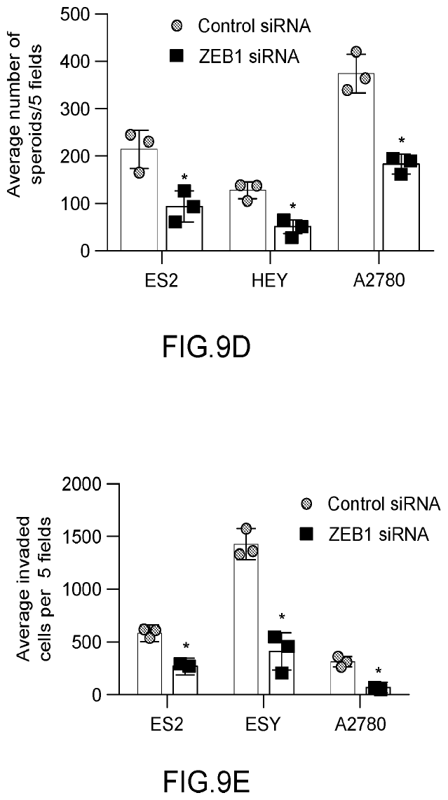

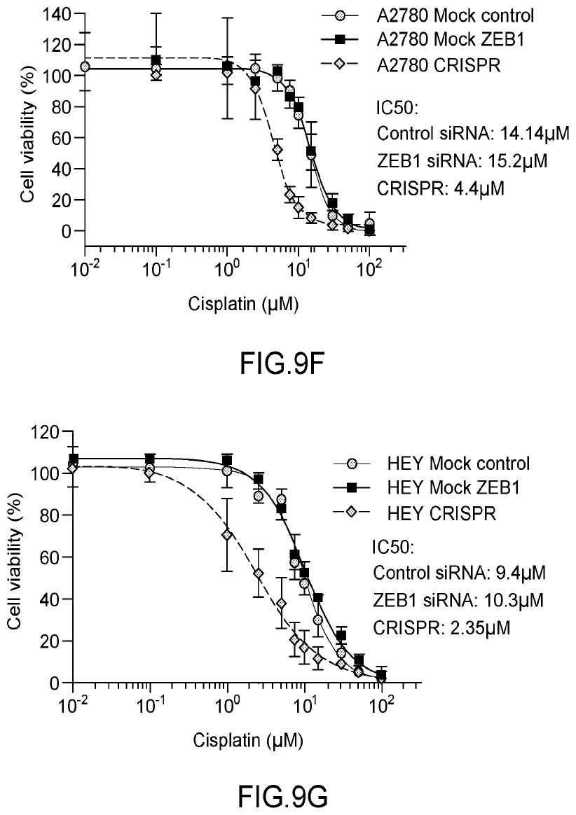

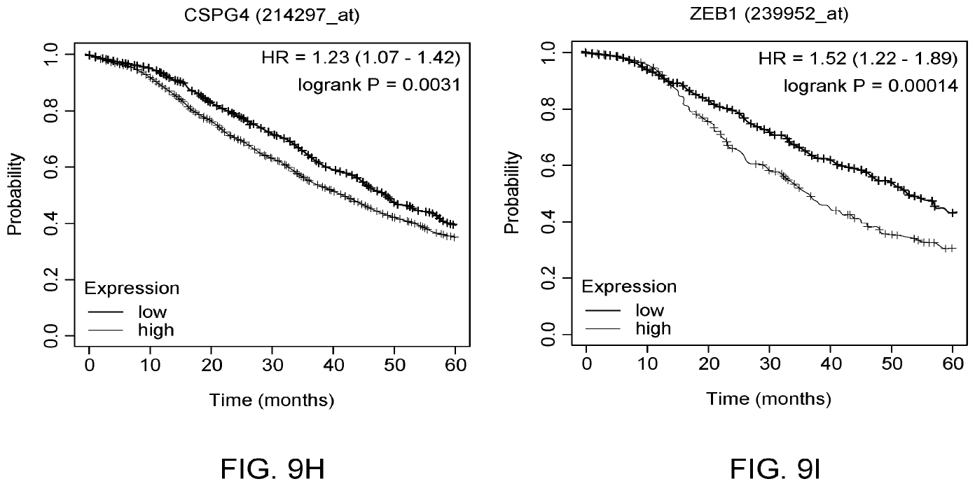

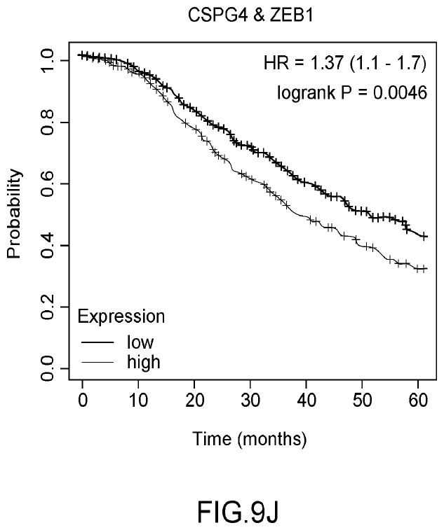

To explore this further, the impact of CSPG4 expression was evaluated on several mesenchymal/epithelial markers in the three EOC cell lines (FIG. 9A). CRISPR/Cas9 driven loss of CSPG4 expression decreased the expression of multiple mesenchymal biomarkers, including vimentin and the mesenchymal transcription factors SNAI2, ZEB1, while promoting increased expression of the epithelial biomarker Claudin-1 (FIG. 9A).

The relationship between CSPG4 and ZEB1 levels was focused on since ZEB1 expression has previously been associated with the development of a mesenchymal phenotype and poor outcome in EOC patients. Inhibiting ZEB1 expression using RNAi (FIG. 9B) had no inhibitory impact on CSPG4 levels, indicating that ZEB1 expression is downstream of CSPG4. Furthermore, CSPG4 stimulated ZEB1 expression is mediated by FAK activation, since ZEB1 expression is inhibited by a small molecule inhibitor of FAK activation, PND-1186 (FIG. 9C). Inhibiting ZEB1 limits both spheroid formation (FIG. 9D) and invasion (FIG. 9E), which is consistent with studies demonstrating that spheroid formation is associated with a mesenchymal transition. However, limiting ZEB1 expression had no detectable impact on cisplatin sensitivity (FIGS. 9F and 9G), indicating that some of the pro-oncogenic effects of CSPG4 are independent of ZEB1 transcriptional activation. Aggregated survival analysis of multiple ovarian cancer patient datasets show that elevated transcript expression of both CSPG4 and ZEB1, either individually or in combination, is significantly associated with decreased overall survival at 5 years (FIG. 9 H-J), supporting the conclusion that both biomarkers can function in concert to promote recurrence and relapse.

### Example 6—Anti-CSPG4 Antibody Decreases EOC Invasion and Spheroid Formation by Inhibiting CSPG4 Activated FAK-ZEBI Pathway

While multiple structural/functional domains of the extracellular portion of the CSPG4 core protein have been identified, many of these sites (e.g., collagen and growth factor binding sites, chondroitin sulfate attachment sites, laminin G-domain) map to membrane distal regions of the core protein. See, e.g., Price, et al., Pigment Cell Melanoma Res. 24(6):1148-57 (2011); Tamburini et al., FASEB J. 33(3):3112-28 (2019). By contrast there is less known of the potential functional importance of domains in the core protein that are membrane proximal. To examine the importance of this juxtamembrane protein region (DE) in CSPG4 on EOC cells, a recombinant fragment of the CSPG4 core protein containing this region (Q1538-N2221, see FIG. 1A) was purified from a eukaryotic expression system and used in the production of mouse monoclonal antibodies. An antibody clone (7H5A2) was identified that specifically recognizes CSPG4 on the cell surface (See FIGS. 1B and 1C). The antibody is antagonistic to CSPG4 function and similar to CSPG4 deletion, inhibited activation of FAK and ZEB1 expression in all three cell lines (FIG. 10A). The antibody also significantly inhibited EOC invasion (FIG. 10B) to a greater extent than the anti-CSPG4 antibody 763.74 (see, e.g., Luo et al., J Immunol 2006; 176:6046-54). Antibody 763.74 has previously been shown to impact CSPG4 mediated invasion of other tumor types, but in contrast to 7H5A2, it recognizes a distinct membrane distal epitope on the core protein. The 7H5A2 antibody also significantly inhibited spheroid formation and EOC cell survival of CSPG4 expressing mock transfected EOC cells (FIG. 10C). While genetically editing CSPG4 expression also inhibited spheroid formation, CRISPR/Cas9 edited cells remained largely viable at the conclusion of the methylcellulose culture (FIG. 7E), indicating that the 7H5A2 antibody has additional cytotoxic mechanisms independent of its impact on CSPG4 stimulated tumor cell growth.

### Example 7—Anti-CSPG4 Generated Against the D3 Domain of the CSPG Core Protein Stimulate Antibody-Dependent Cell-Mediated Cytotoxicity (ADCC) of EOC Cells

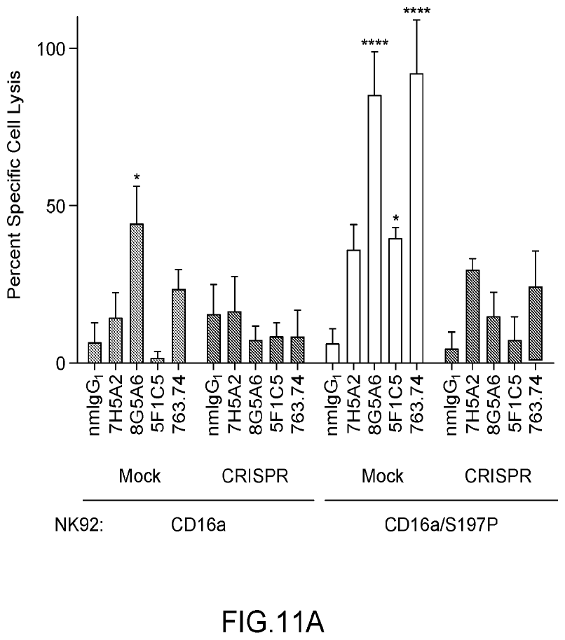

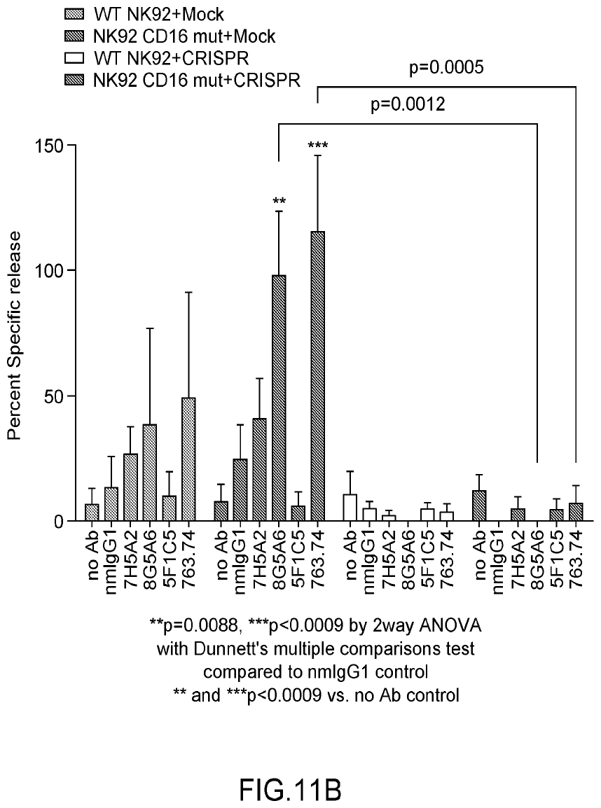

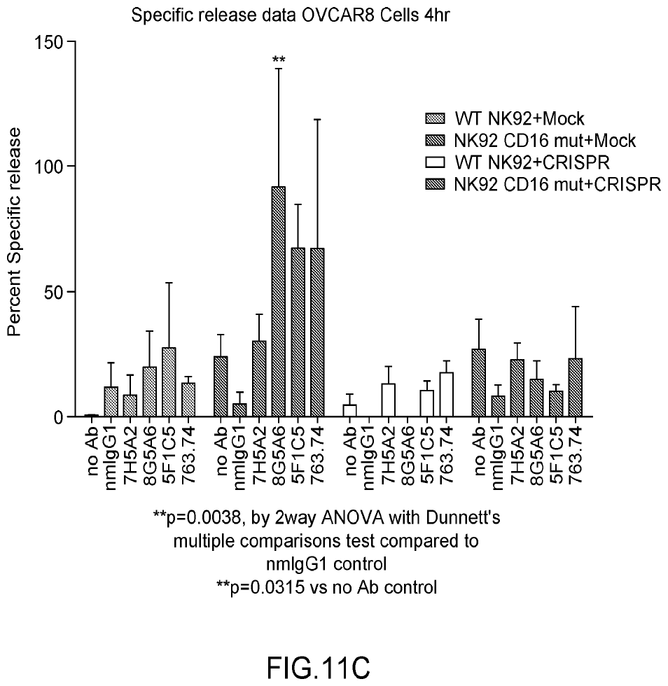

An additional anti-CSPG4 antibody, 8G5A5, was identified using the methods of Example 6. A preliminary screen was done using NK cells that express either a wild type CD16a or a mutated hyperactive CD16a/S197P that enhances ADCC by resisting proteolytic cleavage following NK cell activation. HEY Mock and CRISPR cells were treated with the indicated anti-CSPG4 monoclonal antibodies or normal mouse IgG1 (nmIgGl). The indicated NK92 cell lines were added at a 1:1 effector:target ratio and ADCC determined at 4 hrs using the DELFIA EuTDA cytotoxicity assay following the manufacturer's protocol. The results demonstrate that antibody 8G5A6 can mediate specific ADCC of CSPG4 positive EOC cells mediated by either NK cells expressing wild-type CD16a or CD16a/S197P (FIG. 11A). The experiment was repeated and the results are shown in FIG. 11B. The experiment also was performed with OVCAR8 ovarian cancer cells (FIG. 11C), which are a model for high grade serous ovarian cancer, clinically the most common in women. Again, antibody 8G5A6 was able to mediate specific ADCC of CSPG4 positive cells.

In summary, IHC data from this EOC patient cohort demonstrates that high levels of CSPG4 within tumors are an independent risk factor for poor overall survival. CSPG4 expression promotes tumor expansion in vivo and promotes tumor cell invasion, cisplatin resistance and spheroid formation in vitro. CSPG4 does not signal directly on its own, but rather functions as a co-receptor/plasma membrane scaffold that enhances the intensity and duration of multiple stimulated oncogenic pathways. A longer duration of signal transduction activation can lead to nuclear changes that impact on the transcriptome, as we have shown for CSPG4-mediated prolonged activation of Erk, which causes a shift to a mesenchymal transcriptome in melanoma cells and promotes their tumorigenic potential. Thus, localized elevations of CSPG4 levels in subpopulations of EOC cells, may sustain tumor cell subpopulations that have enhanced oncogenic signaling leading to increased growth, survival and/or invasive potential. This is consistent with the tumor growth data in vivo, which link CSPG4 expression to significantly enhanced tumor expansion compared to CRISPR/Cas9 deleted counterparts. It is also important to note that although high CSPG4 expression levels in EOC tumors negatively impact patient survival, the staining pattern in these tumors is heterogeneous. Since CSPG4 expression is stimulated by microenvironmental changes in hypoxia or inflammatory mediators such as TNFα, heterogeneous CSPG4 expression may be related to these, or additional microenvironmental factors in the expanding tumor.

One consequence of CSPG4 expression is that it stimulates a mesenchymal shift in the phenotype of EOC cells, which is associated with spheroid formation and subsequent intraperitoneal metastasis to other organs such as omentum. More globally, TCGA gene expression data demonstrate the elevated levels of CSPG4 are associated with an EMT signature, and the current data from multiple EOC cell lines link CSPG4 to expression of ZEB1, a mesenchymal transcription factor in EOC and other tumors. As a transcriptional regulator, ZEB1 represses the expression of multiple epithelial genes while it stimulates the expression of genes that are associated with an invasive, mesenchymal phenotype. Since TCGA analysis indicates co-expression of CSPG4 and ZEB1 is associated with poor overall survival, the two may function in concert to promote metastasis as evidenced by their impact on spheroid formation and invasion. Furthermore, CSPG4 expressing tumor cells are also more resistant to cisplatin, suggesting that CSPG4 expressing tumor cells may form a therapy-resistant tumor cell reservoir that promotes relapse following initial standard of treatment.

CSPG4 promotes spheroid formation and invasion by activating FAK and enhancing ZEB1 expression. This is consistent with data from other cell model systems (including fibroblasts isolated from FAK-null animals) which linked FAK activation to ZEB1 expression. As described herein, a well characterized inhibitor of FAK activation limits ZEB1 expression and an anti-CSPG4 specific antibody described herein inhibits FAK activation, ZEB1 expression and tumor cell invasion/spheroid formation. These data are consistent with reports linking CSPG4 function in tumor cells to functionally activating β1 integrins and FAK. Thus, these data support a model in which cell surface CSPG4 interacting with components of the microenvironment (specific ECM components or various growth factors) can enhance mesenchymal transition of EOC cells.

However, while limiting CSPG4 expression causes a significant decrease in platinum IC50s, specifically inhibiting ZEB1 expression has no impact on these values. This leads us to conclude that CSPG4 enhances cisplatin sensitivity by a ZEB1-independent mechanism(s). As a multifunctional transmembrane signaling node, CSPG4 functions to alter the activation by multiple extracellular stimuli (e.g., TGFβ, FGF, HGF) and depending on the cellular context, it can activate multiple oncogenic pathways (e.g., FAK, MAPK, PI3K, NF-kB) in tumor cells. Since reduced cisplatin sensitivity in standard of care therapy may impact the survival of resistant clones of EOC tumor cells, it will be important to further define the mechanisms by which CSPG4 alters the response to this therapy. One approach is to rescue CSPG4 null cells using several well-defined CSPG4 structural mutants to identify domains that fail to reverse the loss of cisplatin sensitivity. This approach may lead to enhanced targeting by identifying CSPG4 domains that limit cisplatin sensitivity by mechanism(s) that are coincident with, or independent of, regulating ZEB1 expression.

While the current data indicate that CSPG4 may directly reduce tumor cell sensitivity to cisplatin, as described herein, CSPG4 in the larger context of tumor tissues may also impact poor outcome in EOC patients by contributing to cell adhesion-related mechanisms associated with environmental mediated drug resistance (EMDR). The concept that underlies EMDR is that adherent tumor cell subpopulations, which initially resist therapy, can form a reservoir of resistant cells that may undergo additional mutations that are responsible for therapy resistant relapse following standard of care. This is analogous to, but distinct from, the hypothesis that therapy resistant cancer initiating stem cells are responsible for therapy failure. Numerous cell adhesion related mechanisms (e.g., regulated by integrin and growth factor/cytokine mediated pathways) can function to promote survival in the absence of transcriptomic profiles that regulate cancer initiating stem cells. Mesenchymal shifts in EOC, driven by factors such as TGF-β, are associated with a collagen remodeling fibrotic gene signature that correlates with metastasis and poor overall survival. The fibrotic signature associated with mesenchymal EOC includes elevated type VI collagen, a major ECM ligand for CSPG4 and elevations in type VI collagen in the tumor parenchyma are associated with decreased EOC patient survival. Those studies demonstrated that EOC cells adherent on type VI collagen coated surfaces exhibited increased resistance to cisplatin in vitro. The potential clinical impact is that localized CSPG4/ECM interactions may cause the formation of therapy-resistant adherent ‘niches’ consisting of deeply embedded EOC populations that may evade detection following standard of care surgical debulking.

Targeting CSPG4 with antibodies that bind the juxtamembrane region of the CSPG4 core protein effectively inhibits ZEB1 expression, limits CSPG4-mediated invasion and promotes apoptosis of EOC cells in spheroids. Thus, targeting this region of CSPG4 can be used to limit metastasis in patients with EOC and thus improve patient outcome.

### Example 8—Exemplary Anti-CSPG4 Antibody

This Example shows the amino acid sequences of the heavy chain and the light chain (kappa) variable domain of the 7H5A2 (mouse) antibody are provided. The CDRs are in bold and underlined text within the variable domain. The sequences of each CDR and framework region, and the nucleotide sequences encoding each of the heavy and light chains also are provided.

Heavy Chain Variable Region Sequence
H-
(SEQ ID NO: 41)
CAGGTTCAGCTGCAGCAGTCTGGGGCTGAACTGGTGAGGCCTGGGTCCTCAGTGAAGATT
TCCTGCAAGGCTTCTGGCTTTGCATTCAGTAACTACTGGATGAACTGGGTGAAGCAGAGG
CCTGGACAGGGTCTTGAGTGGATTGGACAGATTTTTCCTGGAGATGGTGATACTAACTAC
AATGGAAAGTTCAAGGGTAAAGCCACACTGACTGCAGACAAATCCTCCAGCACAGCCTA
CATGCAGCTCATTAGCCTAACATCTGAGGACTCTGCGGTCTATTTCTGTGTTAGAGAGAG
CGACTGGGGCCGAGGCACCACGCTCACAGTCTCCTCA
Translated Polypeptide:
(SEQ ID NO: 8)
QVQLQQSGAELVRPGSSVKISCKASGFAFSNYWMNWVKQRPGQGLEWIGQIFPGDG
DTNYNGKFKGKATLTADKSSSTAYMQLISLTSEDSAVYFCVRESDWGRGTTLTVSS
CDR1
(SEQ ID NO: 1)
GFAFSNY
CDR2
(SEQ ID NO: 2)
FPGDGD
CDR3
(SEQ ID NO: 3)
ESD
Framework 1
(SEQ ID NO: 4)
QVQLQQSGAELVRPGSSVKISCKAS
Framework 2
(SEQ ID NO: 5)
WMNWVKQRPGQGLEWIGQI
Framework 3
(SEQ ID NO: 6)
TNYNGKFKGKATLTADKSSSTAYMQLISLTSEDSAVYFCVR
Framework 4
(SEQ ID NO: 7)
WGRGTTLTVSS
Light Chain Variable Region Sequence
K-
(SEQ ID NO: 42)
GATATCCAGATGACACAGACTACATCCTCCCTGTCTGCCTCTCTGGGAGACAGAGTCACC
ATCAGTTGCAGTGCAAGTCAGGGCATTAACAATTATTTAAACTGGTATCAGCAGACACCA
GATGGAACTGTTAAACTCCTGATCTTTTACACATCAAGTTTACAGTCAGGAGTCCCATCA
AGGTTCAGTGGCAGTGGGTCTGGGACAGATTATTCTCTCACCATCAGCAACCTGGAACCT
GAAGATATTGCCACTTACTATTGTCAGCAGTATAGTAAGCTTCCATTCACGTTCGGCTCG
GGGACAAAGTTGGAAATAAAACGG
Translated Polypeptide:
(SEQ ID NO: 16)
DIQMTQTTSSLSASLGDRVTISCSASQGINNYLNWYQQTPDGTVKLLIFYTSSLQSGVP
SRFSGSGSGTDYSLTISNLEPEDIATYYCQQYSKLPFTFGSGTKLEIKR
CDR1
(SEQ ID NO: 9)
SASQGINNYLN
CDR2
(SEQ ID NO: 10)
YTSSLQS
CDR3
(SEQ ID NO: 11)
QQYSKLPFT
Framework 1
(SEQ ID NO: 12)
DIQMTQTTSSLSASLGDRVTISC
Framework 2
(SEQ ID NO: 13)
WYQQTPDGTVKLLIF
Framework 3
(SEQ ID NO: 14)
GVPSRFSGSGSGTDYSLTISNLEPEDIATYYC
Framework 4
(SEQ ID NO: 15)
FGSGTKLEIKR

### Example 9—Exemplary Anti-CSPG4 Antibody

This Example shows the amino acid sequences of the heavy chain and the light chain (kappa) of the 8G5A6 (mouse) antibody are provided. The CDRs are in bold and underlined text within the variable domain. The sequences of each CDR and framework region, and the nucleotide sequences encoding each of the heavy and light chains also are provided.

Heavy Chain Variable Region Sequence
H-
(SEQ ID NO: 43)
GAGGTCCAGCTGCAGCAGTCTGGACCTGACCTGGTGAAGCCTGGGGCTTCAGTGAAGAT
ATCCTGCAAGGCTTCTGGTTACTCATTCACTGTCTACTACATGAACTGGGTGAAGCAGAG
CCATGGAAAGAGCCTTGAGTGGATTGGACGTATTGATCCTAACGATGGTGGTGCTAGTTA
CAACCAGAAGTTCAAGGGCAAGGCCATATTAACTGTTGACAAGTCATCCGGCACAGCCTA
CATGGAGCTCCGCAGCCTGACATCTGAGGACTCTGCGGTCTATTACTGTGCAAGAAGGG
ATTACTACGGTAGTAAGGACTACTGGGGCCAAGGCACCACTCTCACAGTCTCCTCA
Translated polypeptide:
(SEQ ID NO: 24)
EVQLQQSGPDLVKPGASVKISCKASGYSFTVYYMNWVKQSHGKSLEWIGRIDPND
GGASYNQKFKGKAILTVDKSSGTAYMELRSLTSEDSAVYYCARRDYYGSKDYWG
QGTTLTVSS
CDR1
(SEQ ID NO: 17)
KASGYSFTVYYMN
CDR2
(SEQ ID NO: 18)
RIDPNDGGAS
CDR3
(SEQ ID NO: 19)
ARRDYYGSKDY
Framework 1
(SEQ ID NO: 20)
EVQLQQSGPDLVKPGASVKISC
Framework 2
(SEQ ID NO: 21)
WVKQSHGKSLEWIG
Framework 3
(SEQ ID NO: 22)
YNQKFKGKAILTVDKSSGTAYMELRSLTSEDSAVYYC
Framework 4
(SEQ ID NO: 23)
WGQGTTLTVSS
Light Chain Variable Region Sequence
K-
(SEQ ID NO: 44)
GACATTGTGCTGACCCAATCTCCAGCTTCTTTGGCTGTGTCTCTAGGGCAGAGGGCCACC
ATCTCCTGCAAGGCCAGCCAAAGTGTTGATTATGATGGTGATAGTTATATGAACTGGTAC
CAACAGAAACCAGGACAGCCACCCAAACTCCTCATCTATGCTGCATCCAATCTAGAATCT
GGTATCCCAGCCAGGTTTAGTGGCAGTGGGTCTGGGACAGACTTCACCCTCAACATCCAT
CCTGTGGAGGAGGAGGATGCTGCAATCTATTACTGTCAGCAAAGTAATGAGGATCCGTG
GACGTTCGGTGGAGGCACCAAGCTGGAAATCAAACGG
Translated polypeptide:
(SEQ ID NO: 32)
DIVLTQSPASLAVSLGQRATISCKASQSVDYDGDSYMNWYQQKPGQPPKLLIYAASN
LESGIPARFSGSGSGTDFTLNIHPVEEEDAAIYYCQQSNEDPWTFGGGTKLEIKR
CDR1
(SEQ ID NO: 25)
KASQSVDYDGDSYMN
CDR2
(SEQ ID NO: 26)
YAASNLES
CDR3
(SEQ ID NO: 27)
QQSNEDPWT
Framework 1
(SEQ ID NO: 28)
DIVLTQSPASLAVSLGQRATISC
Framework 2
(SEQ ID NO: 29)
WYQQKPGQPPKLLI
Framework 3
(SEQ ID NO: 30)
GIPARFSGSGSGTDFTLNIHPVEEEDAAIYYC
Framework 4
(SEQ ID NO: 31)
FGGGTKLEIKR

### Example 10—CSPG4 TriKEs and Cancer

This Example demonstrates that CSPG4 TriKEs induce NK-mediated killing of CSPG4 positive cancer cells.

### Methods

### Peripheral Blood Mononuclear Cell Donors and Natural Killer Cell Enrichment

Fresh whole blood was obtained from normal volunteers. Peripheral blood mononuclear cells (PBMCs) were isolated using density-gradient centrifugation with density gradient medium Lymphoprep™ (STEMCELL Technologies, Cambridge, MA, USA). According to the manufacturer's recommendations, Natural killer (NK) cells were enriched by magnetically depleting CD3 and CD19-positive cells using the EasySep™ Human NK Cell Enrichment kit (STEMCELL Technologies, Cambridge, MA, USA).

### Tumor Spheroid Killing Assay

Tumor spheroid killing assays was evaluated in real-time using the IncuCyte SX5-Live Cell Analysis platform. 20,000 GFP-expressing ovarian cancer target cells (OVCAR-8 or SKOV3) were plated in wells of a round bottom ultra-low adhesion 96-well plate (Corning, UK) and allowed to form spheroids for 2 days. 40,000 NK cells magnetically enriched from fresh PBMCs were added with or without 30 nM CSPG4 TriKEs (8G5A6 or 7H5A2) or 3 nM IL-15 to triplicate wells, and plates were incubated for 4 days within the IncuCyte SX5 at 37° C./5% CO₂. Images from three technical replicate wells for each condition were taken every 2 hour for 96 hours using a 1Ox objective lens and then analyzed using IncuCyte™ Basic Software v2018A (Sartorious). Graphed readouts represented target spheroid green fluoresce intensity, normalized to untreated spheroid control wells at each time point.

### Cell Lines

Ovarian cancer cell lines OVCAR-8, SKOV3, OVCAR-3, A2780, MA-148, and OVCAR-5 were cultured in DMEM medium (Mediatech cat #10-013-cv). HEY cells were cultured in RPMI 1640 medium (Gibco cat #11875-093). All culture media was supplemented with 10% fetal bovine serum (Atlanta Biologicals cat #SS11150H, Lot #H1810S) and 1% penicillin/streptomycin (Gibco cat #15140-122) at 37° C./5%CO₂.

### Cell Lysis and Western Blot

The indicated ovarian cancer cell lines were cultured in six-well plates (1.0×10⁵) in a normal growth medium for 48 hours. Cells were lysed in cell lysate buffer (Cell Signaling, Danvers, MA) and 20 μg of protein/sample was fractionated on 4%/7.5% SDS-PAGE and transferred to PVDF membrane for western blot analysis using standard techniques. Membranes were probed with anti-CSPG4 antibody 9.2.27 (Millipore) and anti-a tubulin antibody (Millipore) and appropriate H1RP-conjugated secondary reagents. Bands were visualized by incubation with Pierce ECL western blotting substrate (Thermo Fisher Scientific).

### Results

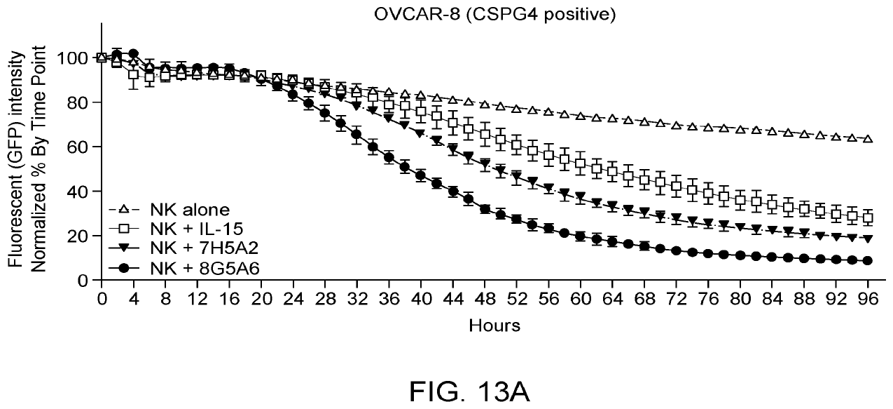

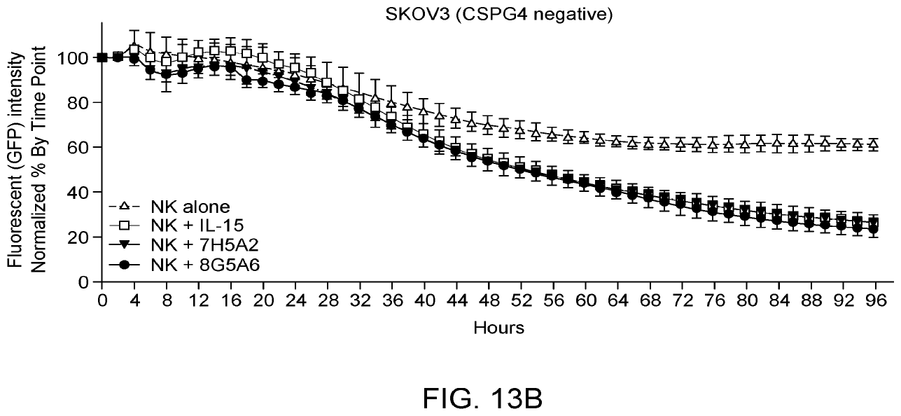

CSPG4 expression was determined in human ovarian cancer cell lines (FIG. 12), and CSPG4 positive and CSPG4 negative treated with NK cells alone, NK cells activated with IL-15, or a CSPG4 TriKE (CSPG4 TriKE 8G5A6 or CSPG4 TriKE 7H5A2. Compared to NK cells alone or NK cells activated only with IL-15 (FIG. 13A), TriKEs based on anti-CSPG4 monoclonal antibodies 7H5A2 or 8G5A6 promoted specific NK-cell mediated killing of spheroids consisting of CSPG4 expressing human ovarian carcinoma cells (OVCAR-8). By contrast, these TriKEs were ineffective at promoting the killing of CSPG4 negative ovarian carcinoma cells over that observed by NK-cells cultured with IL-15 (FIG. 13B).

There results demonstrate that CSPG4 TRiKEs can specifically target CSPG4 expressing ovarian cancers.

### Example 11—Exemplary Embodiments

Embodiment 1. An antibody comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletion, or substitution), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO: 17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).
   Embodiment 2. The antibody of embodiment 1, wherein said antibody comprises the ability to bind to a human CSPG4 polypeptide (SEQ ID NO:33).
   Embodiment 3. The antibody of any one of embodiments 1-2, wherein said antibody comprises said heavy chain variable domain or region of said (i).
   Embodiment 4. The antibody of embodiment 3, wherein said heavy chain variable domain or region comprises an amino acid sequence having at least 90 percent identity to the amino acid sequence set forth in SEQ ID NO:8.
   Embodiment 5. The antibody of any one of embodiments 1-2, wherein said antibody comprises said light chain variable domain or region of said (i).
   Embodiment 6. The antibody of embodiment 5, wherein said light chain variable domain or region comprises an amino acid sequence having at least 90 percent identity to the amino acid sequence set forth in SEQ ID NO:16.
   Embodiment 7. The antibody of any one of embodiments 1-2, wherein said antibody comprises said heavy chain variable domain or region of said (ii).
   Embodiment 8. The antibody of embodiment 7, wherein said heavy chain variable domain or region comprises an amino acid sequence having at least 90 percent identity to the amino acid sequence set forth in SEQ ID NO:24.
   Embodiment 9. The antibody of any one of embodiments 1-2, wherein said antibody comprises said light chain variable domain or region of said (ii).
   Embodiment 10. The antibody of embodiment 9, wherein said light chain variable domain or region comprises an amino acid sequence having at least 90 percent identity to the amino acid sequence set forth in SEQ ID NO:32.
   Embodiment 11. An antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletions or substitution), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO: 17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).
   Embodiment 12. The antigen binding fragment of embodiment 11, wherein said antigen binding fragment comprises the ability to bind to SEQ ID NO:33 or SEQ ID NO:34.
   Embodiment 13. The antigen binding fragment of any one of embodiments 11-12, wherein said antigen binding fragment comprises said heavy chain variable domain or region of said (i).
   Embodiment 14. The antigen binding fragment of embodiment 13, wherein said heavy chain variable domain or region comprises an amino acid sequence having at least 90 percent identity to the amino acid sequence set forth in SEQ ID NO:8.
   Embodiment 15. The antigen binding fragment of any one of embodiments 11-12, wherein said antigen binding fragment comprises said light chain variable domain or region of said (i).
   Embodiment 16. The antigen binding fragment of embodiment 15, wherein said light chain variable domain or region comprises an amino acid sequence having at least 90 percent identity to the amino acid sequence set forth in SEQ ID NO: 16.
   Embodiment 17. The antigen binding fragment of any one of embodiments 11-12, wherein said antigen binding fragment comprises said heavy chain variable domain or region of said (ii).
   Embodiment 18. The antigen binding fragment of embodiment 17, wherein said heavy chain variable domain or region comprises an amino acid sequence having at least 90 percent identity to the amino acid sequence set forth in SEQ ID NO:24.
   Embodiment 19. The antigen binding fragment of any one of embodiments 11-12, wherein said antigen binding fragment comprises said light chain variable domain or region of said (ii).
   Embodiment 20. The antigen binding fragment of embodiment 19, wherein said light chain variable domain or region comprises an amino acid sequence having at least 90 percent identity to the amino acid sequence set forth in SEQ ID NO:32.
   Embodiment 21. The antibody of any one of embodiments 1-10, wherein said antibody is a monoclonal antibody.
   Embodiment 22. The antibody of any one of embodiments 1-10 and 21, wherein said antibody is an scFv antibody.
   Embodiment 23. The antigen binding fragment of any one of embodiments 11-20, wherein said antigen binding fragment is monoclonal.
   Embodiment 24. The antigen binding fragment of any one of embodiments 11-20 and 23, wherein said antigen binding fragment is an Fab.
   Embodiment 25. A chimeric antigen receptor comprising an antigen binding domain, a hinge, a transmembrane domain, and one or more signaling domains, wherein said antigen binding domain comprises an antibody or an antigen-binding fragment of any one of claims 1-24.
   Embodiment 26. The chimeric antigen receptor of embodiment 25, wherein said antigen binding domain comprises a scFv having the ability to bind to a CSPG4 polypeptide.
   Embodiment 27. A cell comprising a chimeric antigen receptor of any one of embodiments 25-26.
   Embodiment 28. The cell of embodiment 27, wherein said cell is a T cell, a stem cell, or an NK cell.
   Embodiment 29. A cell engager comprising a first antigen binding domain, a linker, and a second antigen binding domain, wherein said first antigen binding domain comprises an antibody or an antigen-binding fragment of any one of embodiments 1-24.
   Embodiment 30. The cell engager of embodiment 29, wherein said first antigen binding domain comprises a scFv having the ability to bind to a CSPG4 polypeptide.
   Embodiment 31. The cell engager of embodiment 29, wherein said first antigen binding domain is an IgG having the ability to bind to a CSPG4 polypeptide.
   Embodiment 32. The cell engager of any one of embodiments 29-31, wherein said second antigen binding domain binds to a polypeptide expressed on the surface of T cells.
   Embodiment 33. The cell engager of embodiment 32, wherein said polypeptide expressed on the surface of T cells is a CD3 polypeptide.
   Embodiment 34. The cell engager of any one of embodiments 29-31, wherein said second antigen binding domain binds to a polypeptide expressed on the surface of NK cells.
   Embodiment 35. The cell engager of embodiment 34, wherein said polypeptide expressed on the surface of NK cells is a CD16a polypeptide.
   Embodiment 36. The cell engager of any one of embodiments 29-35, wherein said cell engager comprises a third antigen binding domain.
   Embodiment 37. The cell engager of embodiment 36, wherein said third antigen binding domain binds to a polypeptide expressed on the surface of NK cells.
   Embodiment 38. The cell engager of embodiment 37, wherein said polypeptide expressed on the surface of NK cells is a CD16a polypeptide.
   Embodiment 39. A nucleic acid comprising a nucleic acid sequence encoding at least part of an antibody or an antigen-binding fragment of any one of embodiments 1-24.
   Embodiment 40. The nucleic acid of embodiment 39, wherein said nucleic acid sequence encodes said heavy chain variable domain or region of any one of said (i)-(ii) of embodiment 1.
   Embodiment 41. The nucleic acid of any one of embodiments 39-40, wherein said nucleic acid sequence encodes said light chain variable domain or region of any one of said (i)-(ii) of embodiment 1.
   Embodiment 42. The nucleic acid of any one of embodiments 39-41, wherein said nucleic acid is a viral vector.
   Embodiment 43. The nucleic acid of any one of embodiments 39-41, wherein said nucleic acid is a phagemid.
   Embodiment 44. A nucleic acid comprising a nucleic acid sequence encoding a chimeric antigen receptor of any one of embodiments 25-26 or a cell engager of any one of embodiments 29-38.
   Embodiment 45. The nucleic acid of embodiment 44, wherein said nucleic acid is a viral vector.
   Embodiment 46. The nucleic acid of embodiment 44, wherein said nucleic acid is a phagemid.
   Embodiment 47. A host cell comprising a nucleic acid of any one of embodiments 39-46.
   Embodiment 48. A host cell that expresses a chimeric antigen receptor of any one of embodiments 25-26 or a cell engager of any one of embodiments 29-38.
   Embodiment 49. The host cell of any one of embodiments 47-48, wherein said host cell is a T cell, stem cell, or NK cell.
   Embodiment 50. An antibody-drug conjugate (ADC) comprising an antigen binding domain covalently linked to a drug, wherein said antigen binding domain comprises an antibody or an antigen binding fragment of any one of embodiments 1-24.
   Embodiment 51. The ADC of embodiment 50, wherein said antigen binding domain comprises a scFv having the ability to bind to a CSPG4 polypeptide.
   Embodiment 52. The ADC of embodiment 50, wherein said antigen binding domain is an IgG having the ability to bind to a CSPG4 polypeptide.
   Embodiment 53. The ADC of any one of embodiments 50-52, wherein said drug is selected from the group consisting of calicheamicin, monomethyl auristatin E (MMAE), emtansine (DM1), and an exatecan derivative (Dxd).
   Embodiment 54. A composition comprising an antibody or an antigen binding fragment of any one of embodiments 1-24.
   Embodiment 55. The composition of claim 54, wherein said composition comprises said antibody of any one of embodiments 1-10, 21, and 22.
   Embodiment 56. The composition of claim 54, wherein said composition comprises said antigen binding fragment of any one of embodiments 11-20, 23, and 24.
   Embodiment 57. A composition comprising a cell engager of any one of embodiments 29-38.
   Embodiment 58. A composition comprising a cell of any one of embodiments 27, 28, and 47-49.
   Embodiment 59. A composition comprising an ADC of any one of embodiments 50-53.
   Embodiment 60. The composition of any one of embodiments 54-59, wherein said composition comprises a checkpoint inhibitor.
   Embodiment 61. The composition of embodiment 60, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.
   Embodiment 62. A method of treating a mammal having cancer, wherein said method comprises administering, to said mammal, a composition of any one of embodiments 54-61.
   Embodiment 63. The method of embodiment 62, wherein said mammal is a human.
   Embodiment 64. The method of embodiment 62 or embodiment 63, wherein said cancer is a CSPG4⁺ cancer.
   Embodiment 65. The method of embodiment 71, wherein said CSPG4⁺ cancer is CSPG4⁺ ovarian cancer.
   Embodiment 66. The method of any one of embodiments 62-65, wherein the number of cancer cells within said mammal is reduced following said administering step.
   Embodiment 67. A method for binding a binding molecule to a CSPG4 polypeptide, wherein said method comprises contacting said CSPG4 polypeptide with an antibody or an antigen binding fragment of any one of embodiments 1-24.
   Embodiment 68. The method of embodiment 67, wherein said contacting is performed in vitro.
   Embodiment 69. The method of embodiment 67, wherein said contacting is performed in vivo.
   Embodiment 70. The method of embodiment 67, wherein said contacting is performed within a mammal by administering said antibody or said antigen binding fragment to said mammal.
   Embodiment 71. The method of embodiment 70, wherein said mammal is a human.
   Embodiment 72. A method for binding a binding molecule to a CSPG4 polypeptide, wherein said method comprises contacting said CSPG4 polypeptide with a chimeric antigen receptor of any one of embodiments 25-26, a cell engager of any one of embodiments 29-38, or an ADC of any one of embodiments 50-53.
   Embodiment 73. The method of embodiment 72, wherein said contacting is performed in vitro.
   Embodiment 74. The method of embodiment 72, wherein said contacting is performed in vivo.
   Embodiment 75. The method of embodiment 72, wherein said contacting is performed within a mammal by administering said chimeric antigen receptor, said cell engager, or said ADC to said mammal.
   Embodiment 76. The method of embodiment 75, wherein said mammal is a human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletion, or substitution), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

2. An antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletions or substitution), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

3. The antibody of claim 1, wherein said antibody comprises the ability to bind to a CSPG4 polypeptide.

4. The antibody of claim 1, wherein said antibody comprises said heavy chain variable domain or region of said (i).

5. The antibody of claim 4, wherein said heavy chain variable domain or region comprises an amino acid sequence having at least 90 percent identity to the amino acid sequence set forth in SEQ ID NO:8.

6. The antibody of claim 1, wherein said antibody comprises said light chain variable domain or region of said (i).

7. The antibody of claim 6, wherein said light chain variable domain or region comprises an amino acid sequence having at least 90 percent identity to the amino acid sequence set forth in SEQ ID NO:16.

8. The antibody of claim 1, wherein said antibody comprises said heavy chain variable domain or region of said (ii).

9. The antibody of claim 8, wherein said heavy chain variable domain or region comprises an amino acid sequence having at least 90 percent identity to the amino acid sequence set forth in SEQ ID NO:24.

10. The antibody of claim 1, wherein said antibody or said antigen binding fragment comprises said light chain variable domain or region of said (ii).

11. The antibody of claim 10, wherein said light chain variable domain or region comprises an amino acid sequence having at least 90 percent identity to the amino acid sequence set forth in SEQ ID NO:32.

12. The antibody of claim 1, wherein said antibody is a monoclonal antibody.

13. The antibody of claim 1, wherein said antibody is an scFv antibody.

14. (canceled)

15. (canceled)

16. A chimeric antigen receptor comprising an antigen binding domain, a hinge, a transmembrane domain, and one or more signaling domains, wherein said antigen binding domain comprises an antibody of claim 1.

17. A cell comprising a chimeric antigen receptor of claim 16.

18. The cell of claim 17, wherein said cell is a T cell, a stem cell, or an NK cell.

19. A cell engager comprising a first antigen binding domain, a linker, and a second antigen binding domain, wherein said first antigen binding domain comprises an antibody of claim 1.

20. The cell engager of claim 19, wherein said second antigen binding domain binds to a polypeptide expressed on the surface of T cells.

21. The cell engager of claim 20, wherein said polypeptide expressed on the surface of T cells is a CD3 polypeptide.

22. The cell engager of claim 20, wherein said second antigen binding domain binds to a polypeptide expressed on the surface of NK cells.

23. The cell engager of claim 22, wherein said polypeptide expressed on the surface of NK cells is a CD16a polypeptide.

24-26. (canceled)

27. A nucleic acid comprising a nucleic acid sequence encoding at least part of an antibody of claim 1.

28. A nucleic acid comprising a nucleic acid sequence encoding a chimeric antigen receptor of claim 16.

29. (canceled)

30. (canceled)

31. A host cell comprising a nucleic acid of claim 27.

32. A host cell that expresses a chimeric antigen receptor of claim 16.

33. (canceled)

34. An antibody-drug conjugate (ADC) comprising an antigen binding domain covalently linked to a drug, wherein said antigen binding domain comprises an antibody of claim 1.

35. The ADC of claim 34, wherein said drug is selected from the group consisting of calicheamicin, monomethyl auristatin E (MMAE), emtansine (DM1), and an exatecan derivative (Dxd).

36. A composition comprising an antibody of claim 1.

37-41. (canceled)

42. A method of treating a mammal having cancer, wherein said method comprises administering, to said mammal, a composition of claim 36.

43. The method of claim 42, wherein said mammal is a human.

44. The method of claim 42, wherein said cancer is a CSPG4⁺ cancer.

45. The method of claim 44, wherein said CSPG4⁺ cancer is CSPG4⁺ ovarian cancer.

46. (canceled)

47. A method for binding a binding molecule to a CSPG4 polypeptide, wherein said method comprises contacting said CSPG4 polypeptide with an antibody or an antigen binding fragment of claim 1.

48-52. (canceled)
